(19) United States
(12) Patent Application Publication
(10) Pub. No.: US 2014/0371545 A1
(43) Pub. Date: Dec. 18, 2014

(54) Measurement of Cerebral Physiologic Parameters Using Bioimpedance

(71) Applicants: Shlomi Ben-Ari, Benyamina (IL); Shmuel Marcovitch, Kefar-Saba (IL); Gal Ben-Porath, Tel Aviv (IL)

(72) Inventors: Shlomi Ben-Ari, Benyamina (IL); Shmuel Marcovitch, Kefar-Saba (IL); Gal Ben-Porath, Tel Aviv (IL)

(21) Appl. No.: 14/250,454

(22) Filed: Apr. 11, 2014

Related U.S. Application Data
(60) Provisional application No. 61/811,199, filed on Apr. 12, 2013.

(51) Int. Cl. A61B 5/053; A61B 5/021; A61B 5/00

(52) U.S. Cl. CPC A61B 5/0535; A61B 5/6814; A61B 5/021
USPC 600/301; 600/547

(57) ABSTRACT

Devices and methods are disclosed for detecting and/or monitoring cerebral pathologies. In one embodiment, a cerebro-hemodynamic measurement apparatus is disclosed that includes at least one processor. The at least one processor is configured to receive, via at least one sensor, at least one signal associated with a brain of a subject. The at least one processor is configured to determine, based on the at least one signal, a change in cerebral blood volume caused by a cardiac pulsation. The at least one processor is configured to determine, based on the at least one signal, a change in intracranial pressure due to cardiac pulsation. The at least one processor is also configured to estimate mean intracranial pressure based on changes in the cerebral blood volume, changes in the intracranial pressure, and a compliance indicator.

### RELATED APPLICATIONS

This application claims the benefit of priority under 35 U.S.C. §119(e) of U.S. Provisional Application No. 61/811,199, filed Apr. 12, 2013, the entirety of which is incorporated herein by reference.

### TECHNICAL FIELD

The instant disclosure describes, among other things, mechanisms for detecting and/or monitoring cerebral pathologies.

### BACKGROUND

Cerebral pathologies may lead to temporary brain damage injury, permanent brain damage injury, or death. Examples of cerebral pathologies include ischemic and hemorrhagic strokes, traumatic brain injury (TBI) and vasospasm. Symptoms of these cerebral pathologies often include increased intracranial pressure (ICP). For example, when brain tissue is injured, the injured tissue may develop edema and hemorrhage, both resulting in an increased ICP. To prevent additional brain damage, one may monitor the ICP by inserting a pressure probe into the cranial space. This is an invasive procedure typically involving drilling through the skull (usually at an un-affected area), inserting the probe through the drilled hole, and securing the probe with a nut to the skull or by tunneling a catheter through the scalp. This invasive method typically involves risks associated with insertion of a probe into healthy brain tissue or the ventricular space and risks of infection by an invasive probe.

A non-invasive method and apparatus may be used to measure and monitor ICP and additional intracranial physiological parameters that may be clinically useful for diagnosing strokes, trauma, and other conditions that can affect the functioning of the brain. These parameters may include, for example, cerebral blood volume, cerebral blood flow, cerebral perfusion pressure, vascular cerebral autoregulation functioning and cerebral edema status.

One way to monitor or detect ICP and additional intracranial physiological parameters may include physically inserting a probe into the cerebrospinal fluid or into the parenchyma, angiography, computed tomography angiography (CTA), perfusion computed tomography (PCT), transcranial doppler ultrasound (TCD), positron emission tomography (PET), and magnetic resonance imaging (MRI) and angiography (MRA). Some non-invasive methods for detecting or monitoring ICP and additional intracranial physiological parameters may require, for example, machines for carrying out CT, PCT, PET, and/or MRI procedures. In some instances, the lack of continuous monitoring, the cost of these machines, their limited mobility, and/or their significant expense per use, may limit their usefulness in situations in which regular, continuous, or frequent monitoring of intracranial physiological characteristics may be desirable.

The foregoing description is merely exemplary for providing general background and is not restrictive of the various embodiments of systems, methods, devices, and features as described and claimed.

### SUMMARY OF A FEW ASPECTS OF THE DISCLOSURE

Exemplary disclosed embodiments may include devices and methods for receiving and analyzing impedance plethysmography (IPG) signals representing bioimpedance. More specifically, they may include apparatuses for receiving and analyzing signals and outputting information for estimating physiological brain conditions.

In one embodiment consistent with the present disclosure, a cerebro-hemodynamic measurement apparatus is provided. The cerebro-hemodynamic measurement apparatus may include at least one processor configured to receive, via at least one sensor, at least one signal associated with a brain of a subject, determine based on the at least one signal, a change in cerebral blood volume from a cardiac pulsation, determine, based on the at least one signal, a change in intracranial pressure due to the cardiac pulsation, determine a compliance indicator from a static portion of the at least one signal, and estimate a mean intracranial pressure based on the change in cerebral blood volume, the change in intracranial pressure, and the compliance indicator.

In another embodiment consistent with the present disclosure, a cerebro-hemodynamic measurement apparatus is provided. The cerebro-hemodynamic measurement apparatus may include at least one processor configured to send at least one signal to a first pair of electrodes located on a first portion of a head of a subject, receive at least one IPG signal from a second pair of electrodes located on a second portion of a head of a subject, extract at least one cross IPG waveform corresponding to the first and second portions of the head of the subject from the IPG signal, and estimate a mean ICP based on changes in the at least one cross IPG waveform.

In yet another embodiment consistent with the present disclosure, a cerebro-hemodynamic measurement apparatus is provided. The cerebro-hemodynamic measurement apparatus may include at least one processor configured send signals to at least one pair of electrodes attached to a carrier configured to fit on a head of a subject, receive at least one impedance plethysmography signal associated with a brain of the subject; and estimate a level of damage to at least one of a brain or blood brain barrier using the impedance plethysmography signal.

In still another embodiment consistent with the present disclosure, a cerebro-hemodynamic measurement apparatus is provided. The cerebro-hemodynamic measurement apparatus may include at least one processor configured to receive, via at least a pair of electrodes, at least one signal associated with a brain of a subject, extract at least one impedance waveform from the at least one signal associated with the brain of the subject, and determine an occurrence of vasospasm based on the at least one impedance waveform.

### BRIEF DESCRIPTION OF THE DRAWINGS

The accompanying drawings, which are incorporated in and constitute a part of this specification, together with the description, serve to explain the principles of the embodiments described herein. In the drawings:

FIG. 1 provides a diagrammatic representation of an exemplary IPG measurement apparatus consistent with disclosed embodiments;

FIG. 2 provides a diagrammatic representation of major cerebral arteries;

FIG. 3 provides a diagrammatic representation of exemplary bioimpedance signal pathways in the brain of a subject consistent with disclosed embodiments;

FIG. 4. provides a diagrammatic representation of IPG measurement apparatus hardware consistent with disclosed embodiments;

FIG. 5a provides a diagrammatic representation of an exemplary intracranial pressure waveform;

FIG. 5b provides a diagrammatic representation of an exemplary impedance magnitude waveform, recorded simultaneously to the intracranial pressure waveform, consistent with disclosed embodiments;

FIG. 5c provides a diagrammatic representation of an exemplary impedance phase waveform, recorded simultaneously to the intracranial pressure FIG. 2 illustrates the ICP waveform of a healthy brain;

FIG. 6a provides a diagrammatic representation of an intracranial pressure waveform obtained from a healthy brain under normal conditions;

FIG. 6b provides a diagrammatic representation of an intracranial pressure waveform obtained from a pathological brain;

FIG. 6c provides a diagrammatic representation of an intracranial pressure waveform obtained from a brain under elevated intracranial pressure conditions;

FIG. 6d provides a diagrammatic representation of an intracranial pressure waveform obtained from a brain with a high level of edema, or fluid buildup;

FIG. 7 diagrammatically illustrates a brain compliance curve;

FIG. 8 is a graph illustrating diastolic values of intracranial pressure and arterial blood pressure during respiratory cycles; and

FIG. 9 illustrates an exemplary tissue bioimpedance model.

FIG. 10 illustrates an exemplary graph of edema history;

FIG. 11 illustrates IPG waveforms recorded from a patient experiencing vasospasm; and

FIG. 12 illustrates IPG waveforms recorded from a patient after receipt of vasospasm treatment.

### DETAILED DESCRIPTION

Reference will now be made in detail to exemplary embodiments as with reference to the accompanying drawings. In some instances, the same reference numbers will be used throughout the drawings and the following description to refer to the same or like parts. These embodiments are described in sufficient detail to enable those skilled in the art to practice the disclosed embodiments and it is to be understood that other embodiments may be utilized and that changes may be made without departing from the scope of the disclosed embodiments. The following detailed description, therefore, is not to be interpreted in a limiting sense.

Unless otherwise defined, all technical and/or scientific terms used herein have the same meaning as commonly understood by one of ordinary skill in the art to which the embodiments pertains. Although methods and materials similar or equivalent to those described herein can be used in the practice or testing of the embodiments, exemplary methods and/or materials are described below. In case of conflict, the patent specification, including definitions, will control. In addition, the materials, methods, and examples are illustrative only and are not intended to be necessarily limiting.

Exemplary disclosed embodiments may include devices and methods for the reception and analysis of impedance plethysmography (IPG) signals representing bioimpedance. More specifically, they may include apparatuses for receiving and analyzing signals and outputting information for estimating physiological brain conditions. In some embodiments consistent with the present disclosure, the estimated physiological brain conditions may include conditions associated with ICP. In some embodiments, the estimated physiological brain conditions may be conditions associated with a mean value of ICP.

As used herein, the term “mean value of ICP” refers to the average level of intracranial pressure as measured over a time interval of longer than a heartbeat. In some embodiments, the mean value of ICP refers to the average level of intracranial pressure as measured over a time interval corresponding to an integer number of heartbeats, such that pulsatile or dynamic components are averaged out. The time value over which a mean value of ICP is measured may be as short as a single heartbeat, or may stretch over many minutes or hours. The mean value of the ICP may, in fact, be dynamic itself. Due to such factors as edema development, fluid accumulation, and patient consciousness, the mean value of ICP as measured over, for example, one minute, may vary over the course of hours or days. These changes in the mean value of ICP may be characterized by time scales ranging from approximately half an hour to hours or days.

ICP may be determined based on several factors, including cerebral blood volume (CBV), which is affected by cerebral blood flow, edema status (i.e., intra/extracellular fluid buildup), and cerebral spinal fluid (CSF) volume. Thus, in some embodiments, ICP may be estimated and monitored through determining CBV, edema status, and/or CSF volume. Exemplary devices and methods disclosed herein describe means of monitoring, estimating, and determining CBV, edema status, and CSF volume through the usage of IPG.

Impedance plethysmography (IPG), may be used to measure ICP. In IPG measurement of ICP, electrodes placed externally on a patient's scalp, neck, and/or chest may be used to drive current into the patient and measure the resulting voltage. An impedance plethysmography (IPG) measurement apparatus may be used to measure two sets of resulting voltages associated with the right and left hemispheres of the patient or different sections of the head or other parts of the patient body. The IPG measurement apparatus may compare the driven current and the resulting voltage to determine a bioimpedance measurement in the head of the subject. ICP may be determined at least partially by such a bioimpedance measurement.

Embodiments consistent with the present disclosure may include a measurement apparatus for non-invasive intracranial physiological parameters. In one exemplary embodiment, an IPG measurement apparatus may include, for example, support elements such as a headset, headband, or other framework elements to carry or house additional functional elements. Further structures that may be incorporated may include electrodes, circuitry, processors, sensors, wires, transmitters, receivers, and other devices suitable for obtaining, processing, transmitting, receiving, and analyzing electrical signals. An IPG measurement apparatus may additionally include fasteners, adhesives, and other elements to facilitate attachment to a subject's body. As used herein, an intracranial physiological measurement apparatus need not include all such features.

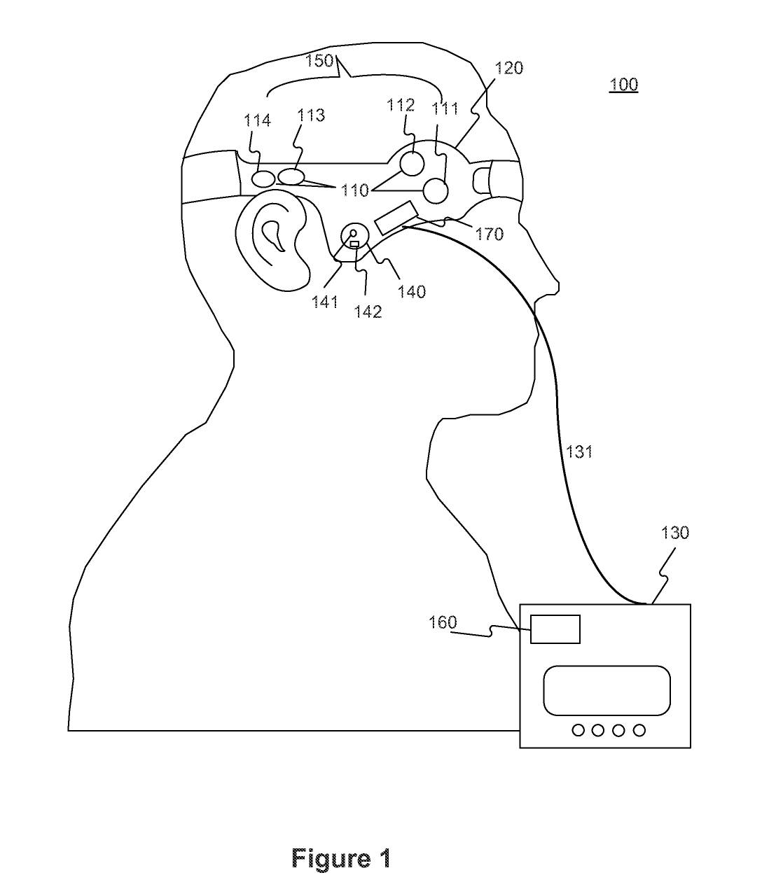

FIG. 1 provides a diagrammatic representation of an exemplary IPG measurement apparatus 100. This exemplary apparatus 100 may include electrodes 110 affixed to a subject's head via a headset 120. Electrodes 110 may be connected to cerebral perfusion monitor 130 via wires 131 (or may alternatively include a wireless connection).

In some exemplary embodiments consistent with the disclosure, an intracranial physiological measurement apparatus may include at least one processor configured to perform an action. As used herein, the term “processor” may include an electric circuit that performs a logic operation on an input or inputs. For example, such a processor may include one or more integrated circuits, microchips, microcontrollers, microprocessors, all or part of a central processing unit (CPU), graphics processing unit (GPU), digital signal processors (DSP), field-programmable gate array (FPGA) or other circuit suitable for executing instructions or performing logic operations. The at least one processor may be configured to perform an action if it is provided with access to, is programmed with, includes, or is otherwise made capable carrying out instructions for performing the action. The at least one processor may be provided with such instructions either directly through information permanently or temporarily maintained in the processor, or through instructions accessed by or provided to the processor. Instructions provided to the processor may be provided in the form of a computer program comprising instructions tangibly embodied on an information carrier, e.g., in a machine-readable storage device, or any tangible computer-readable medium. A computer program may be written in any form of programming language, including compiled or interpreted languages, and it can be deployed in any form, including as a standalone program or as one or more modules, components, subroutines, or other unit suitable for use in a computing environment. The at least one processor may include specialized hardware, general hardware, or a combination of both to execute related instructions. In some embodiments, the at least one processor may include hardware specialized for the task of receiving and interpreting IPG signals; these embodiments are described in more detail below. The at least one processor may also include an integrated communications interface, or a communications interface may be included separate and apart from the at least one processor. The at least one processor may be configured to perform a specified function through a connection to a memory location or storage device in which instructions to perform that function are stored.

Exemplary embodiments of IPG sensors may include various configurations. An IPG sensor may include at least one electrode configured to deliver alternating current and at least one electrode configured to measure a resulting voltage. In some embodiments, an IPG sensor may include two electrodes for current delivery and two electrodes for voltage measurement. In some embodiments, part or all of the at least one voltage receiving electrode and the at least one current delivery electrode may be included in the same physical structure. That is, a single physical electrode may function as both a voltage receiving electrode and as a current delivery electrode. A voltage measurement electrode may be associated with a particular current delivery electrode. A voltage measurement electrode associated with a current delivery electrode may be configured to measure the voltages associated with the current delivered by that particular current delivery electrode. In some embodiments, associated electrodes may be located quite close or in substantially the same place as one another on a patient. In other embodiments, associated electrodes may be located remotely from each other on a patient.

Consistent with some disclosed embodiments, the at least one processor may be configured to receive a signal. As used herein, a signal may include any time-varying or spatially-varying quantity. Receiving a signal may include obtaining a signal through conductive means, such as wires or circuitry; reception of a wirelessly transmitted signal; and/or reception of a signal previously recorded, such as a signal stored in memory. Receiving a signal may further encompass other methods known in the art for signal reception.

At least one processor 160, schematically illustrated in FIG. 1, configured to receive and analyze one or more IPG signals associated with a brain of a subject, may be included in cerebral perfusion monitor 130, as part of exemplary IPG measurement apparatus 100. Processor 160 may be configured to perform all or some of the signal analysis methods described herein, or some of those functions may be performed by a separate processor. Processor 160 may also be configured to perform any common signal processing task known to those of skill in the art, such as filtering, noise-removal, etc. Processor 160 may further be configured to perform pre-processing tasks specific to the signal analysis techniques described herein. Such pre-processing tasks may include, but are not limited to, removal of signal artifacts, such as motion artifacts.

Processor 160 may be configured to receive a signal from one or more electrodes 110, included in exemplary headset 120 of FIG. 1. Electrodes 110 may be arranged singly, in pairs, or in other appropriate groupings, depending on implementation. The electrodes on exemplary headset 120 may be arranged so as to obtain IPG signals. IPG signals may be measured by two sensor sections 150, disposed on the right and left sides of the head to correspond with the right and left hemispheres of the brain, for example. While only one sensor section 150 is shown in FIG. 1, an opposite side of the subject's head might include a similar electrode arrangement. In addition, each sensor section 150 may include one pair of front electrodes, front current electrode 111 and front voltage electrode 112, and one pair of rear electrodes, rear current electrode 114, and rear voltage electrode 113. The distance between the pairs may be adjusted such that a particular aspect of an intracranial physiological condition is satisfied. The electrode configuration depicted in FIG. 1 is only one example of a suitable electrode configuration. Additional embodiments may include more or fewer electrodes 110, additionally or alternatively arranged in different areas of exemplary headset 120. Other embodiments may include electrodes 110 configured on an alternatively shaped headset to reach different areas of the subject's head as compared to the exemplary headset 120.

Pairs of electrodes 110 may include a current output electrode and a voltage input electrode. For instance, front current electrode 111 and front voltage electrode 112 may form an electrode pair. In one embodiment, an output current may be generated by cerebral perfusion monitor 130 and passed between front current electrode 111 and rear current electrode 114. The output current may include an alternating current (AC) signal of constant amplitude and stable frequency in the range of 1 KHz to 1 MHz. An input voltage resulting due to the output current may be measured between front voltage electrode 112 and rear voltage electrode 113. An input voltage may be measured at the same frequency as the output current. A comparison between the output current signal, e.g. a measurement signal, and the input voltage signal, e.g. a response signal, may be used to extract an impedance waveform of the subject. More specifically, a magnitude of the bioimpedance may be computed as a ratio of the input voltage signal amplitude to the output current amplitude signal, and a phase of the bioimpedance may be computed as the phase difference by which the output current signal leads the input voltage signal. Additional impedance components may be computed from the output current signal and the input voltage signal, or from the bioimpedance magnitude and phase, as required.

In one exemplary embodiment, four IPG sensors may be attached to the patient, each sensor including four electrodes. One IPG sensor may be attached to the patient neck or chest, and may obtain and provide a signal from blood entering into the cranial space. This signal may be used as a reference. A second IPG sensor may be attached to the upper portion of the scalp and may obtain and provide a signal correlated with brain movement close to the upper portion of the skull and from the blood leaving the cranial cavity. In addition, one IPG sensor may be attached to each side of the head of a patient and may obtain and provide signals corresponding to brain movement in the inside of the cranial cavity, blood volume, and flow in the main arteries and/or inside brain tissue, for each hemisphere of the brain

An IPG signal may also include output current at more than a single AC frequency. The output current may include a set of predefined frequencies and amplitudes, for example in the range of 1 KHz to 1 MHz, with detection of the measured voltage at all of the frequencies or a part of the frequency range.

Blood and fluid flow into and out of the head, and more specifically, the brain, may result in changes in the cranial bioimpedance characterized by the IPG signal measured by electrodes 110. Bioimpedance changes may correlate with blood volume and blood pressure in the head and brain, as well as the volumes and pressure of other fluids within the brain. The cardiac cycle, respiration cycle, and ICP slow-waves cycle affect the volume and pressure of both blood and other fluids in the brain. In general, because blood and other fluids have a relatively low impedance when compared with tissue found in the head, higher blood or fluid volume results in a lower impedance magnitude. Impedance changes associated with differing blood and fluid volume and pressure within the brain may also cause variations in the frequency response of the brain impedance. Comparing bioimpedance measurements at different frequencies may provide additional information indicative of hemodynamic characteristics.

The exemplary headset 120 may include further devices or elements for augmenting bioimpedance measurements or for performing measurements in addition to bioimpedance measurements, such as an additional sensor or sensors 140. In one embodiment, additional sensor 140 may include, for example, a light emitting diode 141 and a photo detector 142 for performing Photo Plethysmography (PPG) measurements either in conjunction with or as an alternative to bioimpedance signal measurements. The exemplary headset 120 may further include various circuitry 170 for signal processing or other applications and may include the capability to transmit data wirelessly to cerebral perfusion monitor 130 or to other locations. In an additional embodiment, cerebral perfusion monitor 130 may be integrated with headset 120. Although illustrated in the example of FIG. 1, additional sensor 140 and circuitry 170 may be omitted.

Exemplary headset 120 may include various means for connecting, encompassing, and affixing electrodes 110 to a patient's head. For example, headset 120 may include two or more separate sections that are connected to form a loop or a band that circumscribes the patient's head. Any of these aspects, including bands, fasteners, electrode holders, wiring, hook-and-loop connector strips, buckles, buttons, clasps, etc. may be adjustable in order to fit a patient's head. Portions of exemplary headset 120 may be substantially flexible and portions of the exemplary headset 120 may be substantially inflexible. For example, electrode-including portions of exemplary apparatus 120 may be substantially inflexible in order to, among other things, substantially fix electrodes 110 in specific anatomical positions on the patient's head. In addition to or in the alternative, other portions, such as bands or connectors holding the exemplary headset 120 to a patient's head, may be substantially flexible, elastic and/or form fitting.

Any portion of exemplary headset 120 may be specifically designed, shaped or crafted to fit a specific or particular portion of the patient's anatomy. For example, portions of exemplary headset 120 may be crafted to fit near, around or adjacent to the patient's ear. Portions of exemplary headset 120 may be specifically designed, shaped or crafted to fit the temples, forehead and/or to position electrodes 110 in specific anatomical or other positions. Portions of the exemplary headset 120 may be shaped such that electrodes 110 (or other included measurement devices) occur in specific positions for detecting characteristics of blood and fluid flow in the head or brain of the patient. Examples of such blood flow may occur in any of the blood vessels discussed herein, such as the arteries and vasculature providing blood to the head and/or brain, regardless of whether the vessels are in the brain or feed the brain.

Exemplary headset 120 may include features suitable for improving comfort of the patient and/or adherence to the patient. For example exemplary headset 120 may include holes in the device that allow ventilation for the patient's skin. Exemplary headset 120 may further include padding, cushions, stabilizers, fur, foam felt, or any other material for increasing patient comfort.

As mentioned previously, exemplary headset 120 may include one or more additional sensors 140 in addition to or as an alternative to electrical or electrode including devices for measuring bioimpedance. For example, additional sensor 140 may include one or more components configured to obtain PPG data from an area of the patient. Additional sensors 140 may comprise any other suitable devices, and are not limited to the single sensor illustrated in FIG. 1. Other examples of additional sensor 140 include devices for measuring local temperature (e.g., thermocouples, thermometers, etc.) and/or devices for performing other biomeasurements and for devices for measuring movement and positioning of the patient (e.g., accelerometers and/or inclinometers).

Exemplary headset 120 may include any suitable form of communicative mechanism or apparatus. For example, headset 120 may be configured to communicate or receive data, instructions, signals or other information wirelessly to another device, analytical apparatus and/or computer. Suitable wireless communication methods may include radiofrequency, microwave, and optical communication, and may include standard protocols such as Bluetooth, WiFi, etc. In addition to, or as an alternative to these configurations, exemplary headset 120 may further include wires, connectors or other conduits configured to communicate or receive data, instructions, signals or other information to another device, analytical apparatus and/or computer. Exemplary headset 120 may further include any suitable type of connector or connective capability. Such suitable types of connectors or connective capabilities may include any standard computer connection (e.g., universal serial bus connection, firewire connection, Ethernet or any other connection that permits data transmission). Such suitable types of connectors or connective capabilities may further or alternatively include specialized ports or connectors configured for the exemplary apparatus 100 or configured for other devices and applications.

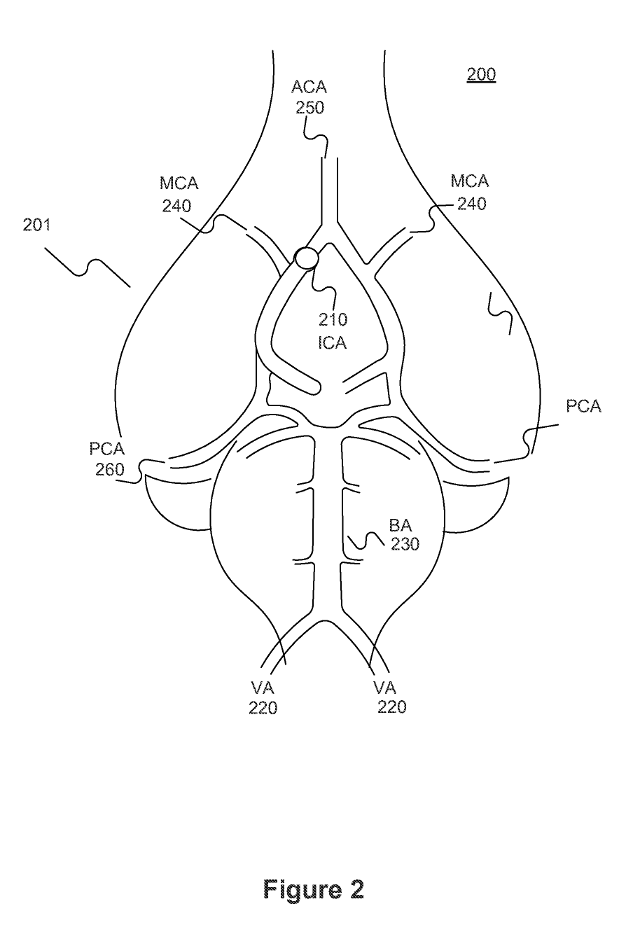

FIG. 2 provides a diagrammatic representation of major features of a cerebral vasculature 200. The cerebral vasculature in FIG. 2 is viewed from below the brain, with the top of the page representing the front of a subject. The blood supply to the brain 201 comes from four main arteries traversing the neck. The larger two are the right and left internal carotid arteries (ICA) 210, in the front part of the neck. The vertebral arteries (VA) 220 are located in the back of the neck and join to form the basilar artery (BA) 230. The internal carotid arteries and the basilar arteries are connected by Posterior Communicating Artery (not shown) and Anterior Communicating Artery (not shown) to form the Circle of Willis (COW). In an ideal patient, the COW is a network of connected arteries that allows blood supply to the brain 201 even when one or more of the feeding arteries is blocked.

The main arteries that supply blood to the brain 201 are the Middle Cerebral Arteries (MCAs) 240, Anterior Cerebral Arteries (ACAs) 250, and Posterior Cerebral Arteries (PCAs) 260.

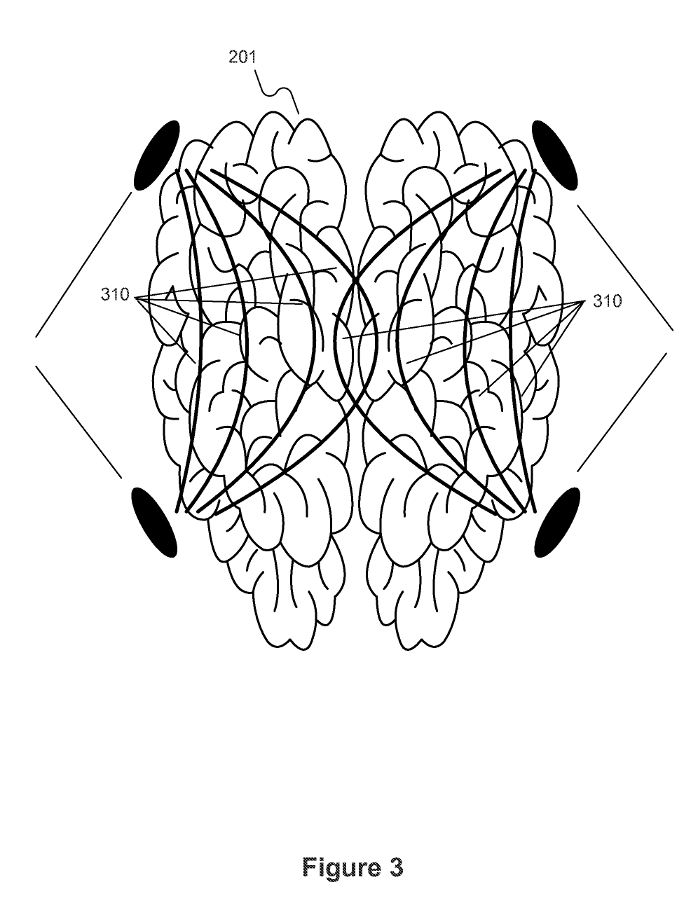

FIG. 3 provides a diagrammatic representation of exemplary impedance signal pathways 310 in the brain 201 of a subject. The exemplary configuration illustrates multiple signal pathways 310 through each of the right and left brain hemispheres. The multiple signal pathways extend between electrodes 110 affixed to the head of a subject via headset 120. The impedance of the signal pathways 310 may be influenced by the presence or absence of blood along the pathway, because blood has a relatively low impedance. At least some of the signal pathways 310 may be coincident with brain vasculature. Signal properties may thus be measured that are indicative of hemodynamic characteristics, such as pressure, blood flow, or volume, in the blood vessels of the brain 201, and/or CSF volume. Changes in bioimpedance may thus be indicative of changes in pressure, blood flow, or blood volume, in the brain 201 and/or CSF volume. Signal pathways 310 depicted in FIG. 3 are representative of only a small number of an infinite number of pathways which may exist in the general area of signal pathways 310.

In some embodiments consistent with the present disclosure, an IPG signal associated with the brain of the subject may include at least a left hemisphere IPG signal and a right hemisphere IPG signal. A left or right hemisphere IPG signal, as used herein, may include an IPG signal reflective of impedance characteristics of the side of the brain with which it is associated. Left and right hemisphere IPG signals may be obtained from either side of the head, as impedance characteristics of the left hemisphere may be obtained from a location on the right side of a subject's head, and vice versa. An IPG signal relating to a particular side of a subject's brain may also be obtained from other locations, such as on the neck of a subject, where, for example, carotid arteries are located, or from frontal and rear portions of a brain.

According to embodiments consistent with the present disclosure, the IPG waveforms may be used to determine ICP, and, more specifically, mean ICP. As noted above, the ICP may be influenced by three general intracranial associated factors: CBV, edema status, and CSF volumes. The ICP may also be influenced by several cyclical parameters of the body, including but not limited to, the cardiac cycle, the respiration cycle, and the ICP slow-wave cycle corresponding to the body's natural vascular cerebral autoregulation of cerebral blood flow. These three factors may affect the ICP at different time scales. The highest frequency variations in the ICP signal may be associated with the cardiac cycle and the arterial blood pressure changes induced by the heart's beating. At lower frequencies, the influence of the respiration cycle and corresponding changes to intrathoracic pressure may be detected in the ICP. At even lower frequencies, ICP slow-waves or plateau-waves with periods on the order of tens of seconds to several minutes correspond to the reactivity time scale of the vascular cerebral autoregulation mechanism. ICP slow-waves are pressure variations having a period of between approximately twenty seconds and several minutes. ICP slow-waves may be associated with physiological cerebral changes caused by the vascular cerebral autoregulation mechanism.

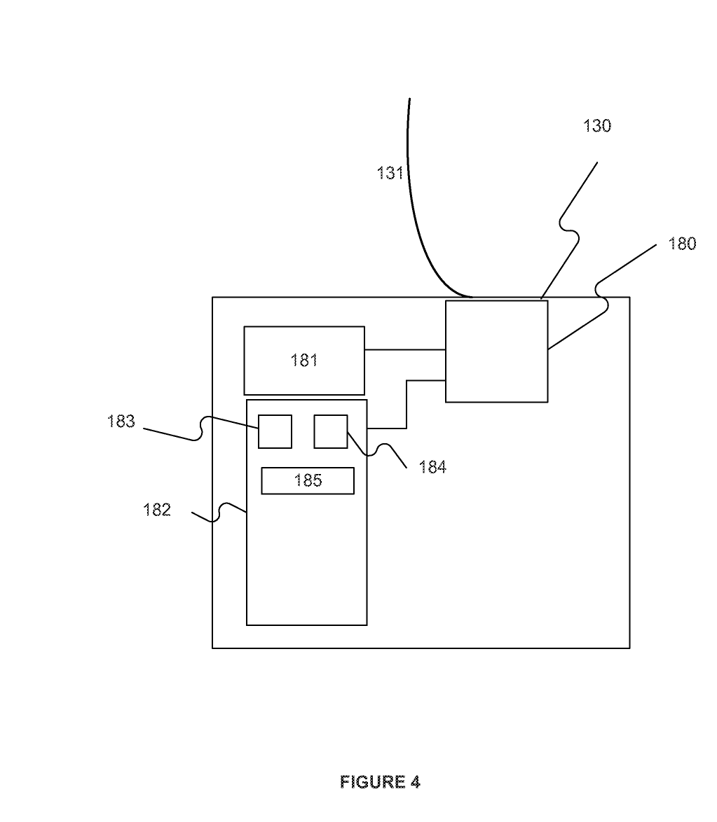

FIG. 4 illustrates additional features of cerebral perfusion monitor 130 consistent with exemplary embodiments of the present disclosure.

A switching unit 180 may be used to rearrange electrode configurations in the headset 120 to obtain IPG signals. For example, a frontal pair of voltage and current electrodes 110 may be used to provide a frontal IPG signal and a rear pair of voltage and current electrodes 110 may be used to provide an intracranial IPG signal. The left/right arrangement and frontal/rear arrangements may be electronically or mechanically switched by switching unit 180. Switching unit 180 may be included as part of processor 160, or may be a separate unit. In another example, the current delivery or voltage measurement electrodes within a sensor may swap roles. In yet another example, a current delivery electrode associated with a particular voltage measurement electrode may be switched. In still another example, electrodes in a different sensor may be designated to perform a new or different function, for example to arrange current delivery or voltage measurement from a different location on the patient. In general, the IPG measurement apparatus may be configured to enable any electrode included within the apparatus, regardless of which sensor it may be included within, to perform any function contemplated herein in association with any other electrode included within the apparatus.

Electrodes may be configured for switching at a very high rate, switching as frequently as every few milliseconds, and may be configured to perform a single role for seconds or minutes at a time.

Through switching, the IPG measurement apparatus can obtain data from different sensor configurations and locations, which may provide additional information on the cerebral status of a patient as compared to conventional fixed sensors or electrodes.

In some embodiments, IPG measurement apparatus 130 may be configured to utilize two or more hardware signal channels to receive IPG signals. In some such embodiments, multiple IPG signals may be measured simultaneously, for example by using a different alternating current frequency in each measurement. Using this technique, the voltage signal obtained from each measurement may be demodulated by one of the hardware signal channels with respect to its corresponding current.

In other embodiments, IPG measurement apparatus 130 may be configured, as described above, to regularly switch between different configurations of operating sensors in order to deliver IPG signals to the multiple hardware signal channels. Each configuration may be defined by a pair of operating sensors, from which signals may be obtained for delivery to and analysis by the multiple signal channels.

In order to minimize or prevent destructive interference in each IPG sensor, alternating current may be delivered at different frequencies to each sensor. In further embodiments, IPG measurement apparatus 130 may analyze not only the signal received by a voltage measuring electrode from an associated current delivery electrode, but also the signal received by a voltage measuring electrode of a non-associated current delivery electrode, i.e. a cross signal.

A cross signal, or cross IPG signal, may be obtained by employing a first pair of electrodes to pass current through the head or brain of the subject, and employing a second pair of electrodes to measure a voltage induced in the head or brain of the subject. In a hemispheric configuration of sensors, including a pair of voltage and current sensors located on each side of the head, for example, a cross signal that measures the voltage induced on one side of the head by the current driven on the other may provide information from a central brain area as the current travels trans-cranially from one hemisphere to the other.

In some embodiments the alternating current may be delivered in frequencies which are varying throughout the course of the measurement, either between at least two fixed frequencies or continuously over a spectrum of frequencies. The comparison of the data received at different frequencies may be used for calibrating the measurement by identifying the dependence of the data on the frequency, with one or more of the clinical parameters which are different between patients or which vary slowly in individual patients.

According to the present disclosure, one or more waveforms may be extracted from any signals received by the at least one processor. Extracted waveforms may include, for example, waveforms representative of impedance components and their change over time. Impedance components may include, for example, the magnitude and phase of the impedance, or the resistive and reactive components of the impedance. Extracted waveforms may also be characterized by various combinations of these components. As used herein, a waveform may be considered “extracted” from an IPG signal if it may be derived from a signal or if it may be determined using the signal.

As described herein, the at least one processor 160 may include both software based and hardware based analysis components. In some embodiments, an exemplary system for performing signal reception and waveform extraction may be implemented by one or more hardware based processors. That is, in some embodiments, signal reception and waveform extraction steps may be performed by a dedicated processor, specifically designed for signal processing, such as a digital signal processor (DSP) or field programmable gate array (FPGA). Because a DSP or FPGA may be configured to implement steps in the following method through hardware configuration, rather than software programming, it may be able to process data at a much higher rate than a software-based method. Such a higher rate of processing may enable complex signals, such as IPG signals described herein, to be processed in real time, at substantially the same rate as their reception. As used herein, the term “real time,” with respect to signal processing, refers to processing of signals that occurs fast enough to keep up with an outside process. Thus, any changes in the physical signal being measured may be reflected in the output data quickly thereafter, within less than five seconds, less than 3 seconds, less than one second, less than a half second, less than one hundred milliseconds, less than fifty milliseconds, or faster. There may be small latencies, between receipt of a signal and output of a processed signal, but one consequence of real time signal processing is that it affords the ability to output processed data at substantially the same rate that data is received, without accumulating a backlog of unprocessed data that increases with time. As described above, an IPG measurement apparatus may include multiple hardware signal channels. A hardware signal channel may include a transmission component 181 and a reception component 182. FIG. 4 illustrates a single hardware signal channel connected to input/output wires 131 via switching unit 180 (which may be controlled to alter the electrode pairs to and from which the hardware signal channel is transmitting and receiving). Input/output wires 131 may carry signals back and forth to headset apparatus 120. It is understood that IPG measurement apparatus 130 may include any number of hardware signal channels as necessary.

Transmission component 181 may be configured to output an electronic signal in a continuous sine wave, square wave or any other periodic continuous wave corresponding at a range of frequencies between 1 KHz and 1 MHz. The system may further be configured to output a single frequency over a time interval before switching to a new time interval, where a time interval may vary in length from milliseconds to minutes. Transmission component 181 may also be configured to multiplex signals of several frequencies together at once for output.

Transmission component 181 component may use any suitable circuit or component configuration for outputting an electronic signal at a desired frequency, including, for example, a phase locked loop circuit configuration providing a continuous sine wave and a digital to analog converter receiving a source signal processed in a digital signal processor (DSP) or field programmable gate array (FPGA) and outputting an analog electronic signal. Transmission component 181 may be configured to output a constant alternating current through the use of a current source and to output a constant alternating voltage. The electronic signal output from transmission component 181 may be delivered to at least one electrode of an IPG sensor in order to provide an IPG measurement.

Reception component 182 of an IPG apparatus may be implemented with analog and digital hardware to obtain the I (in-phase) and Q (quadrature) components of the IPG signal as follows. Reception component 182 for a hardware signal channel may include at least one analog-to-digital converter. A first analog-to-digital converter 183 may be configured to receive a physiological signal by receiving a voltage measured by at least one voltage measurement electrode. Some embodiments may include a second analog-to-digital converter 184 configured to receive a current signal corresponding to a current from at least one current delivery electrode. A current signal may have a voltage corresponding to a current at the at least one current delivery electrode. Current at the at least one current delivery electrode may be measured, for example, by a current meter or by measuring the voltage drop across a known resistor in which the current travels in series. In some embodiments, a signal associated with the current applied to the patient may be obtained in digital form directly from a digital source in transmission component 181, without requiring measurement of current induced in the patient. Reception component may include any number of analog to digital converters as required.

Having received a voltage signal and a current signal, reception component 182 of the hardware signal channel may determine an absolute value of the impedance Z, in an exemplary manner as follows. Analog-to-digital converter 183, and, if included, analog-to-digital converter 184 (and any other analog-to-digital converters that may be included) may sample the analog voltage and current signals at rates as high as 5 MHz, and may have between 16 to 24 bits of resolution. The converted digital voltage signal and digital current signal may then be received by a processing portion 185 of the reception component. A processing portion 185 of reception component 182 may include, as described above, an FPGA or DSP. The digital current signal may then be multiplied, in real time, by a pure sinusoid wave I0 with zero degrees of phase shift (i.e., a sine wave) and second pure sinusoid wave Q0 with 90 degrees of phase shift relative to I0 (i.e., a cosine wave). These multiplications yield IC0 and QC0. Similar multiplications are performed for the received digital voltage signals, yielding IV0 and QV0.

These multiplications have the effect of separating the original signals into two parts, the first of which represents the in-phase portion I, and the second of which represents the quadrapolar portion Q, 90 degrees out of phase from I. The resultant signals also have two spectral components, a first at approximately twice the test frequency, and a second close to zero. The spectral components close to zero correspond to the modulation of the test signal as it passes through the body of the subject.

Next, IC0, QC0, IV0, and QV0, may be low pass filtered to remove the high frequency components, leaving behind the portions corresponding to the body's modulation. This step too may be performed in real time by the dedicated hardware of the processing portion.

A final step performed by the processing portion 185 of the reception component 182 may include signal decimation. The IC0, QC0, IV0, and QV0 signals may be decimated to a sample rate between 20 Hz and 1 kHz, to yield extracted waveforms Ic, Qc, Iv, and Qv. These lower sampling rates are more suitable for software processing. The decimated, extracted current and voltage waveforms, Ic, Qc, Iv, and Qv may then be received by a second at least one processor, for example a CPU, that may be configured to perform further processing based on software instructions. These waveforms, which represent the in-phase and quadrature portions of a signal, may be used to determine a complex impedance waveform \vec{Z}, which may be representative of tissue impedance. Further details regarding methods utilized to analyze the extracted waveforms are described below.

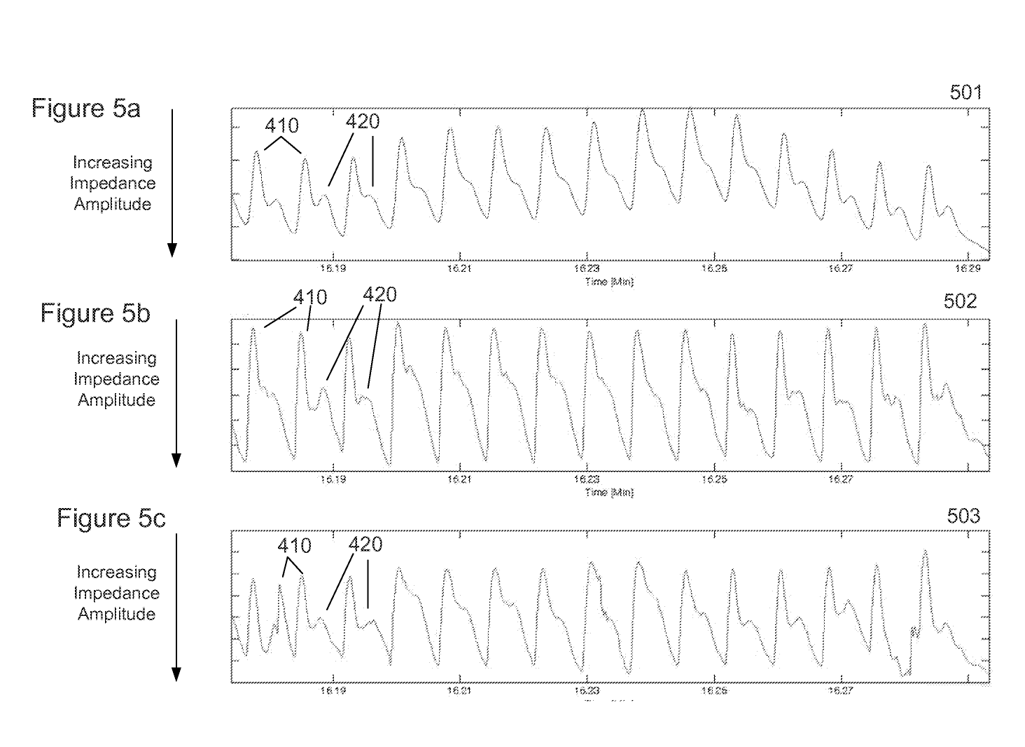

As discussed above, IPG signals may be used to determine ICP levels. This can be illustrated with respect to FIGS. 5a-5c. In FIGS. 5a-c, the impedance magnitude waveform 502 and the phase waveform 503 demonstrate characteristics that correlate with characteristics within the ICP signal 501. FIG. 5a provides a diagrammatic representation of an exemplary ICP signal 501. FIG. 5b provides a diagrammatic representation of an exemplary impedance magnitude waveform 502, recorded simultaneously to the ICP signal 501. FIG. 5c provides a diagrammatic representation of an exemplary impedance phase waveform 503, recorded simultaneously to the ICP signal 501.

For example, all three signals demonstrate first peak P1 410 and second peak P2 420 characteristics. A rise and fall of the mean ICP associated with a respiration cycle can also be seen in the ICP signal 501. Coinciding with the rise and fall of the mean ICP is a similar rise and fall in the height of P2 420 within that signal. Impedance magnitude waveform 502 and impedance phase waveform 503 also demonstrate a rise and fall in the height of P2 420 that coincides with the rise and fall of the mean ICP as shown in ICP signal waveform 501. Thus, information about the mean ICP may be obtained, for instance, from variations in the height of P2 420 within an impedance magnitude waveform 502 or an impedance phase waveform 503. These characteristics are detailed here for exemplary purposes only, as they are readily discernible from mere observation of waveforms 501, 502, and 503. Through additional analysis techniques, as will be discussed in more detail below, additional characteristics may be identified within impedance magnitude waveform 502 or impedance phase waveform 503.

As shown in FIGS. 5a-5c, the IPG waveform closely follows the changes in the ICP waveform, and shows strong similarity to the ICP waveform. Both IPG amplitude and phase waveforms show strong correlations with ICP changes.

The measured IPG waveform may show changes due to relative changes in the blood volume of the tissue through which the IPG current flows and due to additional hemodynamic parameters. The blood volume may vary according to the instantaneous blood pressure and flow during a cardiac cycle, and this change may be captured by the IPG waveform in a cardiac cycle. In clinical testing, dynamic components of IPG waveforms correlate well with dynamic components of ICP waveforms. However, because IPG waveforms measure relative changes in tissue blood volume, mechanical brain pulsation, and CSF pulsatility, additional analysis of the dynamic components of the IPG waveform may be necessary in order to determine, with the assistance of physiological calibration, mean values of ICP.

The dynamic components of ICP waveforms, and their measured IPG analogs, may also be classified by their spectral properties. The highest frequency signal, with the fastest pulsatility, results from the cardiac complexes. Every heart beat drives blood flow to the brain, affecting the measured ICP. At lower frequencies, the signal may be modulated by respiration. Breathing in and out alters the pressure on the jugular veins, which, in turn, alters the pressure required for blood to flow out of the brain, affecting the measured ICP. At still lower frequencies, there are slow waves which correspond to the reactivity time scale of the vascular cerebral autoregulation (CAR) mechanism. The body, and in particular, the brain adjusts blood flow characteristics, through mechanisms such as vasodilation and vasoconstriction; such changes may take tens of seconds up to tens of minutes to be affected.

In some embodiments consistent with the present disclosure, estimating a mean ICP may include eliminating or normalizing dynamic components of an ICP waveform or its representative IPG waveform. After adjusting for the relative amplitudes of pulsatile features of the ICP waveform that correspond to the cardiac complexes, respiratory cycle, and cerebral autoregulation mechanism, the mean value of the ICP remains. From the adjustments necessary to determine a mean ICP value based on an ICP waveform, the adjustments necessary to determine a mean ICP value based on an IPG signal corresponding to an ICP waveform may be determined. All of the factors described above may be useful in monitoring the cranial condition of a patient.

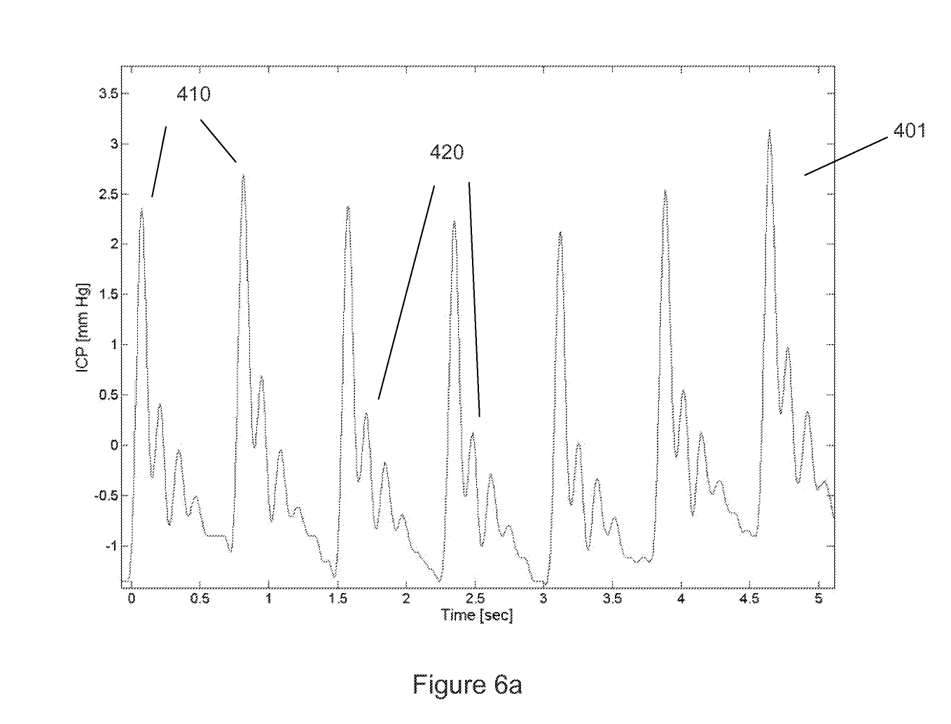

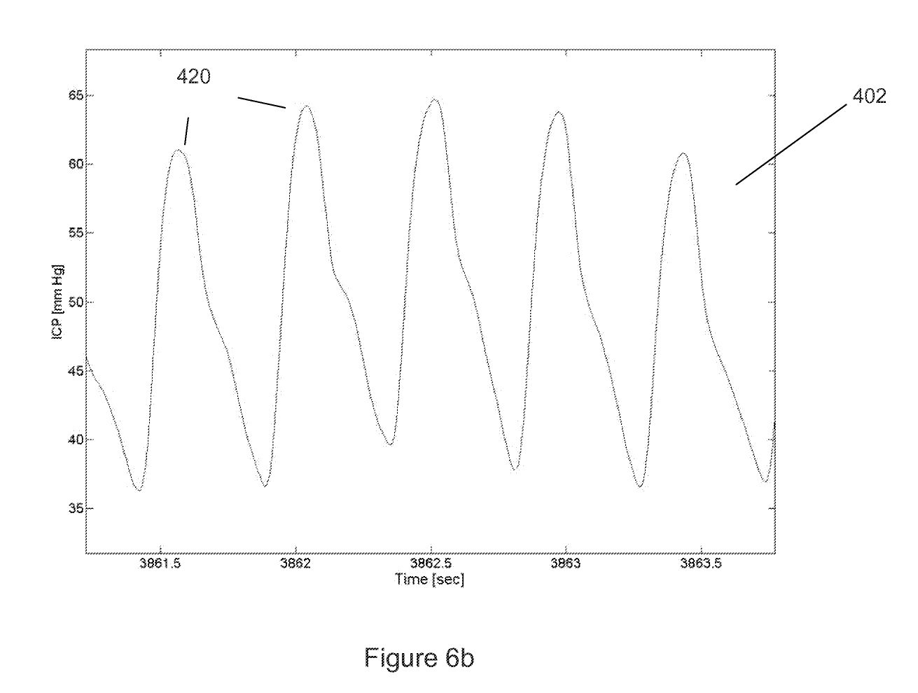

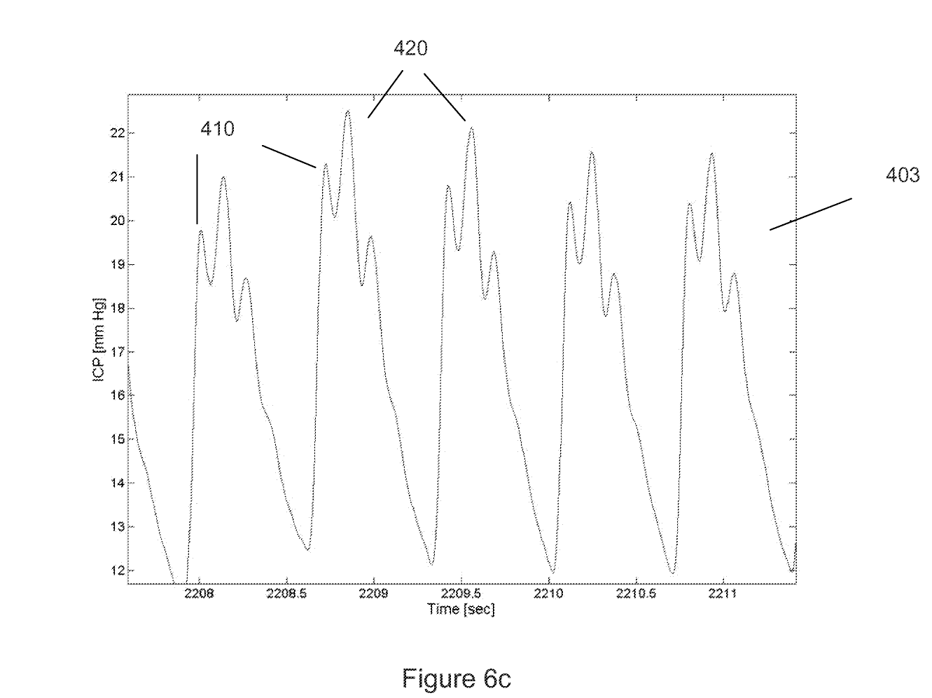

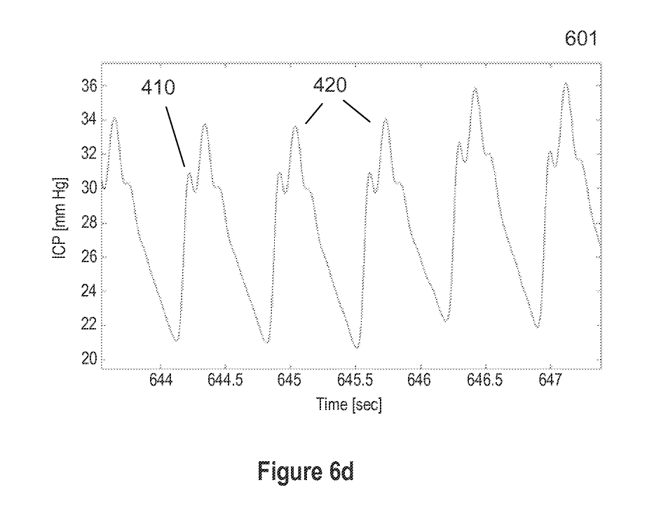

As discussed above, various natural processes, such as cardiac cycles, respiration cycles, and cerebral autoregulation slow-wave cycles affect the volume and pressure of both blood and other fluids in the brain. These can be better understood with respect to FIGS. 6-9.

FIGS. 6a-6d illustrate ICP waveforms obtained through conventional, invasive measures. ICP waveform 401, illustrated in FIG. 6a provides a diagrammatic representation of an ICP waveform obtained from a healthy brain under normal conditions, with an ICP ranging between −1 and 2.5 mm Hg. The first peak (P1) 410 is significantly higher than the second peak (P2) 420 in this waveform. In addition, the signal waveform is characterized by high roughness.

ICP waveform 402, illustrated in FIG. 6b provides a diagrammatic representation of an ICP waveform obtained from a pathological brain, with an ICP ranging between 35 and 60 mm Hg. In ICP waveform 402, P1 410 is not seen, because it is screened by P2 420 which is much higher. In addition, the roughness of the signal waveform is very low—it has only a few characteristic features.

ICP waveform 403, illustrated in FIG. 6c provides a diagrammatic representation of an ICP waveform obtained from a brain under elevated ICP conditions, with the ICP ranging between 12 and 21 mm Hg. In this figure, P2 420 is slightly higher than P1 410, and the roughness is still high.

FIG. 6d illustrates an ICP waveform 601 of a brain with a high level of edema, or fluid buildup. In the illustrated ICP waveform, the height of P2 420 shows a significant increase with respect to the expected level in a healthy brain. Thus, the height of P2 420 may be an indicator of edema level in the brain. As described above, edema level is a contributing factor to ICP elevation, and thus, increased P2 420 height may be indicative of increased ICP mean value in the brain.

Characteristics that are evident in these ICP waveforms vary depending on the condition of the subject's brain. For example, the ratio of a first peak (P1) 410 to a second peak (P2) 420 varies between the signals. In the healthy brain, P1 410 is significantly higher than P2 420. In the pathological brain, P2 420 is expanded in height and width to the point where it screens and obscures P1 410. Finally, in the elevated ICP brain, P1 410 is lower than P2 420. Thus, the ratio of P1 to P2 is an indicator that may correlate with the mean value of the ICP. As another example evident in these waveforms, the roughness of each ICP waveform decreases with an increasing mean ICP. The roughness of a waveform measures the frequency of identifiable variations within the waveform. The P1 to P2 ratio and roughness of the ICP waveforms, as illustrated in FIGS. 6a-c, are exemplary identifiable characteristics in an ICP waveform.

The concavity of the cardiac complex, which may be defined as the relation between the time period the signal is above a certain threshold (e.g., the average of the minimal and maximal value) and the duration of the complex (which equals one divided by the heart rate), may also be indicative of the mean value of ICP. In the healthy brain the concavity ratio is small, as can be seen in FIG. 6a, while in the pathological brain the concavity ratio is larger, as can be seen in FIG. 6b. The concavity ratio is a clinical parameter which may correlate with the mean value of ICP.

Peak to peak (P2P) measurements may also be indicative of a mean value of the ICP. For each cardiac complex in the ICP waveform, the peak to peak measure may be defined as the difference between the maximal value and the minimal value. The cardiac complexes in the ICP signal correspond to the volume of blood entering into the brain each beat, which are defined as Cerebral Stroke Volume (CSV). CSV and Cerebral Blood Flow (CBF) are interlinked, as CBF equals the sum of CSV's over a time period, e.g., of one minute. The peak to peak measure of the cardiac complexes in the ICP signal, thus, may also correlate well with the mean value of ICP. The foregoing represent only exemplary characteristics that may be identified within ICP signals that may be indicative of mean ICP value.

In some embodiments consistent with the present disclosure, a working position on a brain compliance curve may be estimated based on an extracted waveform. As described above, determining a mean ICP may require normalizing for or adjusting for the relative amplitudes of pulsatile features in an ICP or representative IPG waveform. A correlation between the relative measures of the ICP waveform (or representative IPG waveform) and the mean value of the ICP waveform may be determined through an understanding of a compliance curve of the brain. The compliance curve of the brain may be understood as the relationship between brain volume and pressure.

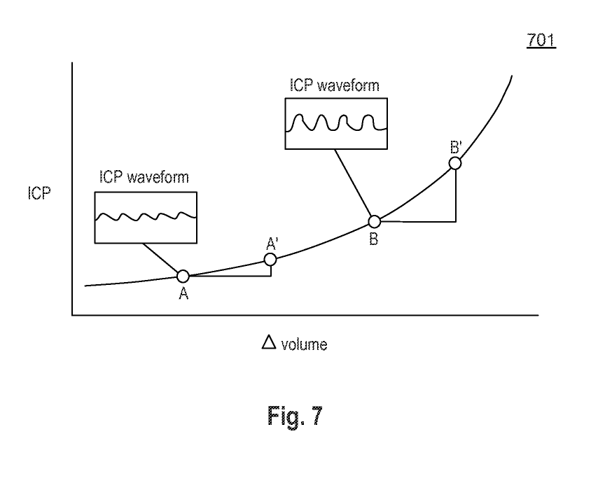

FIG. 7 illustrates a brain compliance curve 701. Brain volume includes brain tissue volume, Cerebral Blood Volume (CBV) and Cerebral Spinal Fluid (CSF). Fast changes in the brain volume may be driven primarily by changes in CBV and CSF. As FIG. 7 illustrates, as the brain volume (x-axis) increases, smaller changes in the brain volume correlate with increasingly larger changes in ICP. Thus, as long as CSV and CSF do not fluctuate too greatly between successive cardiac complexes, the size of variations in the peak to peak measure of the ICP waveform may be indicative of a working position on a brain compliance curve 701, which may further correlate with a mean value of the ICP. For example, a high peak to peak measure of ICP may be indicative of a high CBV (corresponding to B-B′ in FIG. 7), while a low peak to peak measure of ICP may be indicative of a low CBV (corresponding to A-A′ in FIG. 7). This can also be seen in FIG. 4a where the ICP peak to peak measure is 3.5 mm Hg. The peak to peak measure of ICP, therefore, may be an indicator of the mean value of ICP. The peak to peak measure of ICP may be a particularly useful indicator of mean value of ICP when there is a simultaneous indicator of a corresponding change in volume.

Furthermore, the peak to peak measure of an ICP waveform during a single heartbeat complex may also be an indicator of CSF maintenance functioning. As described above, CSF volume maintenance is among the factors that determine mean ICP. In some cases, doctors perform CSF maintenance on patients. However, when CSF is not artificially maintained by physicians, the peak to peak measure of the ICP waveform may be indicative of CSF maintenance functioning. In a situation where CSF fails to flow out of the brain, either due to low CSF availability or blockage of CSF flow, the effect of variations in blood flow on the ICP waveform is larger, as a brain that retains CSF will have a relatively large brain volume, and thus be further to the right on the compliance curve.

In some embodiments consistent with the present disclosure, waveform characteristics extracted from an impedance waveform associated with a patient respiratory cycle may be utilized for estimating a working position on a brain compliance curve. Characteristics of the ICP waveform associated with the respiratory cycle may also be valuable in determining a mean value of ICP. Respiration results in changes to the intrathoracic pressure. Inhalation increases the intrathoracic pressure, thus increasing the external pressure on the jugular vein, which in turn decreases blood outflow from the brain, thereby increasing CBV and hence ICP. ICP measurements taken during a Valsalva maneuver illustrate this. In the Valsalva maneuver, patients may increase their intrathoracic pressure by attempting to expire against a closed airway. During a Valsalva maneuver, measured ICP may increase to values of above 30 mm Hg due to the increase in CBV.

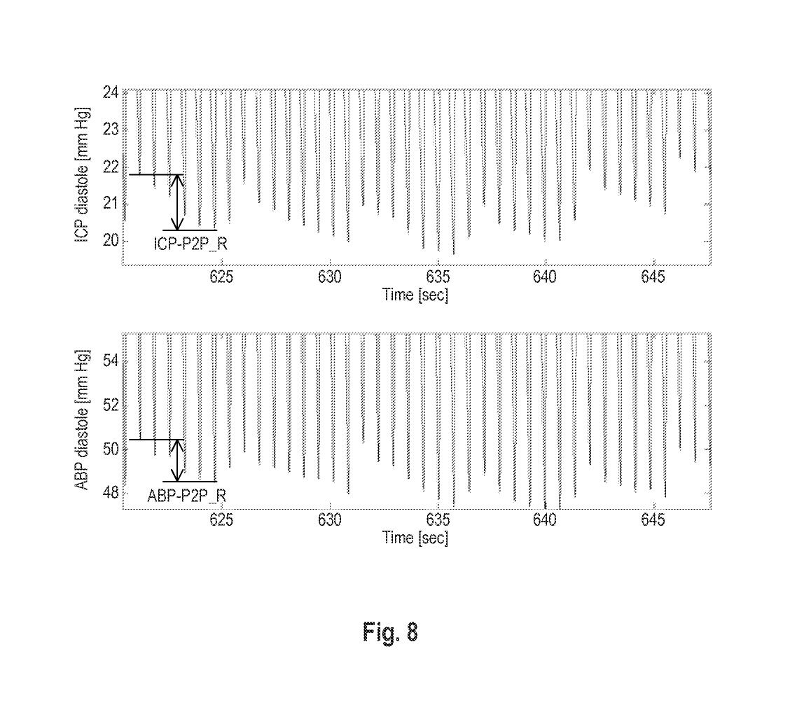

FIG. 8 illustrates diastolic values of ICP and ABP during respiratory cycles. In FIG. 8, the effects of respiratory modulation can be seen in a comparison between ICP and arterial blood pressure (ABP) waveforms. Each downward spike on the graphs shown is the ICP or ABP measure at a diastole portion of the cardiac cycle. As shown, the minimum ICP and ABP show a cyclical pattern over the course of a respiration cycle. The minimum ICP and ABP reach their lowest points during an exhalation phase of a respiratory cycle. As illustrated in FIG. 8, respiratory modulations of the respiratory peak-to-peak measures of ICP and ABP (ICP-P2P_R and ABP-P2P_R, respectively) equal 1.5 mm and 2 mm respectively.

As discussed above, measuring the brain's working position on the compliance curve through ICP may be facilitated by a steady CSV. In some patients, however, the CSV between successive cardiac cycles may not be steady enough to allow for an accurate measurement of the compliance curve working position through ICP. Because the respiratory cycle affects ICP independently of CSV, it may provide a supplemental measure indicative of a brain's position on the compliance curve. ABP, which may be conveniently measured, may be used to provide this supplemental measure. Because ICP is contributed to by factors related to blood flow (CBV) as well as factors not related to blood flow (e.g. CSF level and edema level), a comparison between ICP and ABP may help serve to separate these influences. The difference between changes in blood pressure over a respiratory cycle and changes in ICP over the same respiratory cycle may therefore be indicative of the working position of the brain on the compliance curve. This may be described mathematically as follows. Define CC−R=(ICP−P2P−R)−(ABP−P2P−R). CC−R is a measure indicative of a working location of the brain on the compliance curve. Thus, subtracting the respiratory peak-to-peak measure of arterial blood pressure from the respiratory peak-to-peak measure of intracranial pressure results in a measure indicative of a working position of the brain on the compliance curve.

Additionally, the ratio between a peak to peak ICP measurement during a heartbeat complex at peak inspiration and at peak expiration may be utilized to indicate the current compliance curve working location, through calibration with the ABP signal.

In some embodiments consistent with the present disclosure, characteristics of the ICP waveform associated with an cerebral autoregulation, or slow wave, cycle may be used to determine a mean value of ICP. The pressure reactivity index (PRX), for example, is a measure correlated with the mechanical functioning of the cerebral autoregulation mechanism, and may thus be correlated with a mean value of ICP.

As described above and with respect to FIG. 5a-5c, IPG signals (and extracted IPG waveforms) correlate well with ICP signals. Thus, in situations where directly measured ICP data may be unavailable, for example because the procedure may be too invasive or time consuming, IPG measurements may be used to estimate the various components of ICP waveforms, discussed above, to determine various cerebral parameters.

By way of example only, extracted waveforms representative of impedance components within an IPG signal may be expressed mathematically as follows. As discussed above, a waveform extracted from an IPG signal may be represented by the complex vector 2. As previously described with respect to the cerebral perfusion monitor, a received IPG signal may be broken down into its component current and voltage parts, Ic, Qc, Iv, and Qv. The complex impedance waveform \vec{Z} may be computed from waveforms Ic, Qc, Iv, and Qv, as follows. \vec{Z}=(Iv+j Qv)/[(Ic+j Qc)/R0], where j=\sqrt{−1}, and \vec{Z}=complex impedance of the tissue under study.

Because \vec{Z} represents a complex waveform, it may be represented using the {I,Q} (e.g. in-phase, quadrature) representation, wherein, I=real(\vec{Z}), Q=imag(\vec{Z}). An alternate representation of the impedance may be also given by the amplitude and phase measurements, |Z|=abs(\vec{Z}),

$$ϕ = {tan}^{- 1} ( \frac{Q}{I} ) .$$

Each of the waveforms are time-dependent, where I(t) describes the resistive part of the impedance, Q(t) describes the reactance portion and |Z(t)| characterizes the overall magnitude of the impedance, where all three are measured in units of Ohms. φ(t), the phase angle signal, corresponds to the relation between the reactance and the resistance and may be measured in degrees.

In the analysis of the IPG waveform both the high pulsatility components, for example as the heart complexes and the respiratory modulation, and low pulsatility components, for example, cerebral autoregulation slow-waves and edema development, can be seen in all four measures: I(t), Q(t), |Z(t)|, φ(t).

The waveform of the IPG signal may then be processed with various techniques, such as spectral analysis and mode decomposition techniques to analyze the waveform at varying time scales. For example, waveforms associated with differing physiological processes, such as the cardiac cycle, respiration cycle, or slow-wave cycle, may be extracted from the IPG signal using mode decomposition techniques to eliminate signal elements that occur at frequencies not associated with the appropriate physiological process. The waveform may then be analyzed with respect to the above described pathological indicators and be used to extract the mean value of the ICP and the waveform complex noninvasively. Waveforms for analysis may similarly be extracted from other types of signals, such as ABP signals and ECG signals.

The indicators described above with respect to measuring a mean ICP value, e.g., P1/P2 relation, roughness, concavity measure, P2P measures, CSF functioning, edema indications, and cerebral autoregulation status may be measured or determined using each of the IPG waveforms: I(t), Q(t), |Z(t)|, φ(t). and/or characteristics extracted from these waveforms. Exemplary measurement methods are described in more detail as follows.

In some embodiments consistent with the present disclosure, a processor may be configured to estimate mean ICP from an IPG signal based on a change in cerebral blood volume determined from the signal, a change in ICP determined from the signal, and an indicator from a static portion of the signal. This method of determining ICP values from IPG data may utilize peak to peak measures of the IPG data signal and the compliance curve, and may be performed as follows.

The compliance curve illustrated in FIG. 7 may be defined mathematically as follows: ICP=A e^{bv} where ICP is mean ICP, A and b represent static parameters that vary based on a patient's condition, and V represents the total brain volume. A and b may also be referred to as compliance indicators, as they are factors that describe the compliance curve. As used herein, “static parameters” refers to parameters with rates of change slower than a cardiac pulsation. That is, static parameters are not unchanging, but change at a relatively slower rate than those parameters that change at rates similar to the cardiac cycle. Mathematical exploration of the relationship between mean ICP, A, b, and v demonstrates why the peak to peak measures described above may provide good estimates of ICP. Differentiating the equation defining the compliance curve yields dICP/dV=b*ICP. Of these, dV and dICP may be estimated from dynamic cardiac components in the IPG signal, and b may be estimated from static components of the IPG signal. Static components useful for estimating b may include, for example, the mean value of the static real or imaginary parts of the signal, as well as additional constant factors corresponding to the patient, such as head circumference, age, and gender and other. b, therefore, may represent a compliance indicator determined from a static portion of an IPG signal and external factors.

dV, which represents the change in cerebral volume, correlates well with the change in CBV for each heartbeat, because the other components of cerebral volume do not change on the same time scale as the CBV. As previously discussed, increased blood flow tends to affect the real portions of the IPG signal more strongly than the imaginary portions. Thus, changes in CBV are well correlated with the resistive, or real, portion of the IPG signal. dICP, which represents the change in intracranial pressure, is correlated with tissue deformation that occurs with each heartbeat. The reactive, or imaginary, portion of the IPG signal is associated with changes in cerebral tissue. Thus, dV and dICP may both be estimated from the IPG signal. It should be noted that dV also correlates with the imaginary portion of the IPG signal and dICP correlates with the real portion of the IPG signal, and thus, either or both portions of the IPG signal may be utilized to estimate both of these parameters. Both dV and dICP may be estimated from the peak-to-peak values of the IPG over a cardiac cycle.

Alternatively, changes in CBV may be measured through a hemispherical signal which corresponding to significant cerebral arteries such as the MCA, and changes in pressure (dICP), may be measured through a trans-hemispherical signal, i.e., a cross signal, which may correspond to the capillary reactiveness. dV may also correlate with a trans-hemispherical IPG signal and dICP may also correlate with a hemispherical IPG signal, and thus, either or both of the IPG signals may be utilized to estimate both of these parameters. Both dV and dICP may be estimated from the peak-to-peak values of the IPG over a cardiac cycle.

Various patient conditions, such as age, gender, circumference of head, height, weight, existence of traumatic brain injury, existence of surgical intervention, existence of hemorrhage, existence of edema, pulse rate, and injury side may all influence the value of static parameter b, which may be estimated from static components of the IPG signal after the more dynamic components are removed. In some embodiments, these patient conditions are used to assist in the estimation of indicator b. With estimates of dV, dICP, and b, the equation dP/dV=b*ICP may then yield an estimate of ICP.

Embodiments of the present disclosure may provide for additional means of measuring hemodynamic parameters. For example, in some embodiments consistent with the present disclosure, cardiac stroke volume (CSV) may be measured from IPG data. Changes in the absolute value of the impedance |Z(t)| may correspond to changes in the blood volume inside the brain. Within each cardiac complex, these changes may correspond to the CSV, the amount of blood that enters the brain every beat. This measure is also directly related to CBF, as CBF is, by definition, the sum of the CSV's over a time period, e.g., of one minute.

In some embodiments consistent with the present disclosure, a mean value of ICP may be estimated from mean arterial pressure and CSV. At the frequency of the heart complexes, changes in ICP are mainly due to blood entering into the brain, and thus correlate well |Z(t)| of an IPG waveform. The amount of blood entering the brain depends on Cerebral Perfusion Pressure (CPP), which is equal to CBF multiplied by cerebrovascular resistance (CVR). Cerebrovascular resistance may be estimated from changes in the phase of the impedance waveform, as described in greater detail below. Thus, CPP may be estimated from CSV and CVR. CPP may also be correlated with ICP. That is, ICP=Mean Arterial Pressure (MAP)−CPP. Thus, by using continuous Arterial Blood Pressure (ABP) data to determine mean arterial pressure, measured, for example, from a femoral artery, measuring CSV from an IPG absolute value of impedance, and measuring CVR from an IPG waveform phase, a mean value of the ICP may be estimated.

As discussed above, mean ICP may be also estimated based on an estimation of a working position on a compliance curve. In addition to methods described above, such an estimation may be assisted by estimating edema levels through analysis of impedance phase information. Changes in impedance phase correlate with changes in cerebrovascular resistance. This is at least partially due to the fact that impedance phase is strongly determined by reactive components of the IPG waveform, which reflect changes in tissue structure more strongly than changes in blood flow. Thus, as the cerebral arteries experience geometric modification, e.g. expanding, contracting, stiffening, and softening, thus affecting the CVR, these changes are reflected in the phase portion of the impedance waveform.

In situations where it is only blood volume that changes from one heartbeat to the next, while blood vessels do not encounter any geometrical modifications, the phase portion of the IPG signal may be affected less significantly than the amplitude portion of the IPG signal. This may correspond to a scenario in which there is high pressure on the blood vessels from outside, corresponding to elevated ICP levels due to changes in brain tissue. In contrast, during a Valsalva maneuver, where the ICP is increased due to respiratory effects, the peak to peak measure of φ(t) in each heartbeat complex decreases with increasing ICP much more rapidly than the peak to peak of |Z(t)|. That is, comparing peak to peak measures of the phase portion of an IPG waveform during ICP increases caused by a Valsalva maneuver compared to those ICP increases caused by brain tissue changes demonstrates that the phase portion of the waveform reacts differently to ICP increases caused by brain tissue changes versus ICP increases caused by respiratory effects.

Thus, in some exemplary embodiments consistent with the present disclosure, a working position on a brain compliance curve may be estimated from phase portions of an impedance waveform associated with a respiration cycle. By measuring the peak to peak of φ(t) during a cardiac complex at peak expiration, and the peak to peak of φ(t) during a cardiac complex at peak inspiration, as well as the peak to peak values of respiratory modulation for ABP and IPG amplitude, the working location in the compliance curve may be extracted.

In some exemplary embodiments, a correlation of φ(t) and |Z(t)| may be an indicator of a mean ICP level. In healthy patients, the brain is flexible, and changes due to blood influx are accompanied with vascular geometrical changes. Thus, a timing correlation of φ(t) and |Z(t)| may be relatively low in healthy tissues with low-ICP, while, at higher levels of ICP the two signals may become more synchronized. At higher levels of ICP, when the blood vessels become stiffer due to increased pressure, any changes to the blood vessels (measured by φ(t)) due to the pulsatility of blood flow (measured by |Z(t)|) are more likely to occur with less lag between the blood flow pulse and the vessel change.

In still another exemplary embodiment, mean ICP may be estimated directly from analysis of cross IPG signal data. A carrier, such as exemplary headset 120, may be employed to fit a first pair of electrodes on a first portion of a head of a subject and to fit a second pair of electrodes on a second portion of the head of the subject. In some embodiments, separate carriers may be used for the first and second electrode pairs. At least one processor may be configured to send a signal, such as a current signal, to the first pair of electrodes and to receive an IPG signal, such as a voltage signal, from the second pair of electrodes. A cross IPG waveform may be extracted from the received signal and changes in the cross IPG waveform may be used to estimate mean ICP. The cross IPG waveform, therefore, may correspond to both the first portion and the second portion of the head. The first portion and the second portion of the head may represent, for example, a left side corresponding to a left brain hemisphere and a right side corresponding to a right brain hemisphere, and vice versa.

In some embodiments, a second IPG waveform extracted from a second IPG signal may be used to augment the cross IPG waveform in determining mean ICP. That is, in addition to the cross IPG waveform obtained from two portions of the head, where the voltage and current electrode pairs are spaced away from each other, a standard IPG waveform obtained from a single portion of the head, where the voltage electrodes are placed on the head near the current electrodes may augment the cross IPG waveform in the ICP determination.

The second IPG waveform may be obtained in several ways. For example, an additional sensor pair, each sensor including a voltage electrode and a current electrode may be placed on the head to send the second signal and receive the second IPG signal. In other embodiments, the first pair of electrodes may operate to send both a first signal for generating a cross IPG signal and a second signal for generating a standard IPG waveform. In these embodiments, an additional pair of voltage electrodes may be placed on the head in locations near the first pair of electrodes. In still other embodiments, the first pair of electrodes located on the first portion of the head may send a single signal, functioning as both the at least one signal and the second at least one signal, which may be received by electrodes on a second portion of the head as a cross IPG signal and received by electrodes on the first portion of the head as a standard IPG signal. In yet another embodiment, one of the first or second pairs of electrodes, in addition to generating the cross IPG signal, may be used to both send the second at least one signal and receive the at least one IPG signal. The foregoing electrode combinations do not constitute an exhaustive list, and other suitable combinations may be used to generate a cross IPG signal and a standard IPG signal.

In other embodiments, arterial blood pressure signals and/or non-invasive blood pressure signals may be used to augment the first cross IPG waveform.

In addition to the I/Q and amplitude/phase analysis methods, any suitable mathematical handling of the data prior to extraction of parameters may be utilized. That is, a signal S such that S=function(I, Q, amplitude, phase) may be used, where the function may include mathematical manipulation based on static parameters or based on adaptive parameters which are computed according to the data. Thus, the mathematical manipulation methods may be altered according to the recorded data.

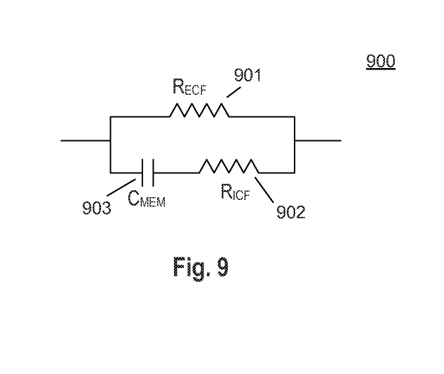

In some embodiments consistent with the present disclosure, edema levels, which may be useful for determining a working location in the compliance curve as well as determining other cerebral parameters, may also be estimated by measuring I(t), Q(t), |Z(t)|, (t), at multiple frequencies. The bioimpedance of tissue may be modeled as illustrated in FIG. 9, as a first resistive element in parallel to a second resistive element and a capacitor. The first resistive element, R_{ECF} 901 may represent the resistance of extracellular fluid, the second resistive element, R_{ICF} 902 may represent the resistance of intracellular fluid, and the capacitor, C_{MEM} 903, may represent the capacitance of cellular membranes. When impedance is measured at a single frequency, the circuit may be analyzed as a single impedance. However, changes in the frequency at which the impedance is measured change the behavior of the capacitor without changing the behavior of the resistors. Thus, by analyzing impedance data at multiple frequencies, an extended picture of the value of each circuit element may be gained. The bioimpedance circuit capacitor may correspond to affects produced by cell membranes, the first resistive element may correspond to affects produced by extracellular fluid (e.g. vasogenic edema), and the second resistive element may correspond to affects produced by intracellular fluid (e.g. cytotoxic edema).

Mathematically, the circuit in FIG. 9 may be represented as follows, where w represents the frequency: Z(w)=R_{ECF}*[R_{ICF}/(j w C_{MEM} R_{ICF}+1)]. Measuring the tissue impedance at multiple frequencies and extracting pulsatile and non-pulsatile parameters from each of the waveforms I(t), Q(t), |Z(t)|, φ(t). at each frequency, multiple equations may be generated. Solving these equations may provide estimates of R_{ECF} 901, the resistance of extracellular fluid, R_{ICF} 902, the resistance of intracellular fluid, C_{MEM} 903, cell membrane capacitance. From these factors, the level of brain edema may be estimated. Estimates of edema may contribute to an estimate of the brain's working position on the compliance curve, as edema is among the factors that contribute to brain volume. Estimates of edema may also provide value for diagnosing other cerebral conditions, as discussed further below.

A method for determining edema levels may operate as follows. Using time-division multiplexing techniques, current may be delivered at frequencies ranging from 10 KHz-1 MHz over a very short time period. In each frequency, approximately 50 wavelengths of current may be delivered. Each frequency may be measured for a period of 0.5-2 milliseconds. Because the range of frequencies are delivered and measured over time scales much shorter than typical physiological changes, the impedance measurements over multiple frequencies are made substantially simultaneously, and are able to capture physiological changes.

Edema estimates generated through IPG analysis may also provide value in the estimation of various types of cerebral edema. Cerebral edema is among the most important factors in mortality and morbidity after traumatic brain injury (TBI). Generally, cerebral edema may be divided into two major types: cytotoxic edema and vasogenic edema.

Cytotoxic edema may develop as a result of changes in brain cell permeability. In this process, extracellular fluid penetrates into brain cells which cause them to swell and eventually die. This process is usually accompanied with large increase in intracranial pressure (ICP), and may lead to brain herniation and death. Vasogenic edema develops as a result of damage to the blood-brain-barrier which results in an increase in the volume of extracellular fluid, and thus a potential increase in ICP.

In ischemic stroke patients, cytotoxic edema tends to dominate. In TBI patients, both vasogenic and cytotoxic edemas may appear at different phases of the secondary injuries. Determining the dominant form of edema at each stage is essential to determining an appropriate treatment for patients.

As described above, IPG signals applied at a range of frequencies may be utilized to estimate cerebral edema levels. Additionally, the techniques described above may also be used to distinguish between the two types of edema and estimate the status of each type of cerebral edema. Biological materials, and in particular cerebral tissues, may be modeled, as described above, by a single resistor R_{ECF} in parallel to a capacitor C_{MEM} and another resistor R_{ICF}, where R_{ECF} corresponds to extracellular fluid, C_{MEM} to cell membrane and R_{ICF} to intracellular fluid. Because intracellular and extracellular fluids serve as good electrical conductors, variations in determined values for R_{ICF} and R_{ECF} may permit both the detection of edema as well as the determination of edema type.

For example, where no edema is present, R_{ICF} and R_{ECF} may both have relatively high values, as the lack of excessive intracellular and extracellular fluid make the conduction of electricity more difficult. In the event of cytotoxic edema, R_{ICF} may be lowered due to the presence of additional intracellular fluid. In the event of vasogenic edema, R_{ECF} may be lowered due to the presence of additional extracellular fluid.

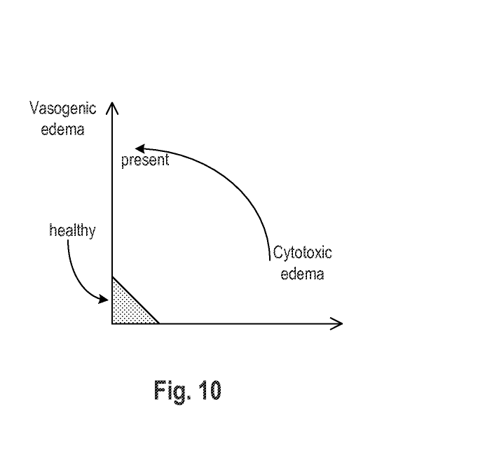

The data may be presented in a two-axis graph which shows the current status of the patient the status of the patient's edema history, as illustrated in FIG. 10. The triangle near point (0,0) corresponds to the healthy regime. The curved arrow exemplifies a scenario in which a patient with mainly cytotoxic edema develops to a situation in which vasogenic edema dominates.

The above described techniques using at least two frequencies may provide valuable information about additional intracranial hemodynamic parameters beyond edema. Different components of a subject's body, e.g., blood, CSF, brain, and white matter, have different impedance spectral properties. By extracting waveform parameters from any two or more impedance signals obtained at two or more frequencies, physiological waveforms of the different cerebral components may be obtained. Additionally, by comparing the timing of events at different frequencies, for example, the time at which the systolic portion of the impedance phase reaches its maximum slope, physiological waveforms of tissues may be extracted with increased accuracy. Thus, a plurality of intracranial hemodynamic parameters, including, for example, ICP level, edema status, cerebral autoregulation functioning, cerebral perfusion, and CSF drainage can be estimated.

Exemplary embodiments of the IPG measurement apparatus consistent with the present disclosure include display devices, alarms, transmitters and other suitable means for conveying patient information to medical personnel. The various physiological and cerebro-hemodynamic parameters discussed herein may be measured and reported to medical personnel through a variety of means. For example, an IPG measurement apparatus may include a screen to display any parameters measured or determined. An IPG measurement apparatus may include wireless or wired network capabilities to inform medical personnel of a patient's condition via e-mail, website, or other network facilitated method.

An IPG measurement apparatus may be configured to inform medical personnel of current patient conditions, e.g. by continuously reporting mean ICP values and or by providing a trend presentation of the ICP values during a longer time interval such as six hours, a day, or a week. In some exemplary embodiments, an IPG measurement apparatus may be configured to determine and report parameter values in a simplified fashion. For example, an IPG measurement apparatus may be configured to determine and report, for example via an alarm, whether a mean ICP surpasses a certain threshold (e.g. 20 mmHg) indicating a dangerous or concerning patient condition. IPG measurement apparatus may also be configured to determine and report mean ICP values in ranges, for example by displaying a green light indicating a safe condition when ICP is below 15 mmHg, a yellow light indicating a potentially harmful or escalating condition when ICP is between 15 and 25 mmHg, and a red light indicating a dangerous condition when ICP exceeds 25 mmHg. Similarly simplified parameter determination and reporting methods may be applied to any of the parameters discussed herein.

In some embodiments consistent with the present disclosure, analysis of impedance waveforms extracted from IPG signals may be used to diagnose and monitor cerebral vasospasm. Cerebral vasospasm, the constriction of cerebral blood vessels, frequently occurs in subjects after they have suffered a hemorrhagic stroke or aneurysm. Vasospasm has the potential to cause significant cerebral damage, but may be difficult to detect. First, the timing of vasospasm, relative to the stroke itself is unpredictable. Vasospasm may occur anywhere from hours to days after a stroke. Second, vasospasm may not cause any outward symptoms until cerebral damage has already occurred. Vasospasm may be treated readily with vasodilation agents, such as nimotop, but such treatments require the successful detection of vasospasm.

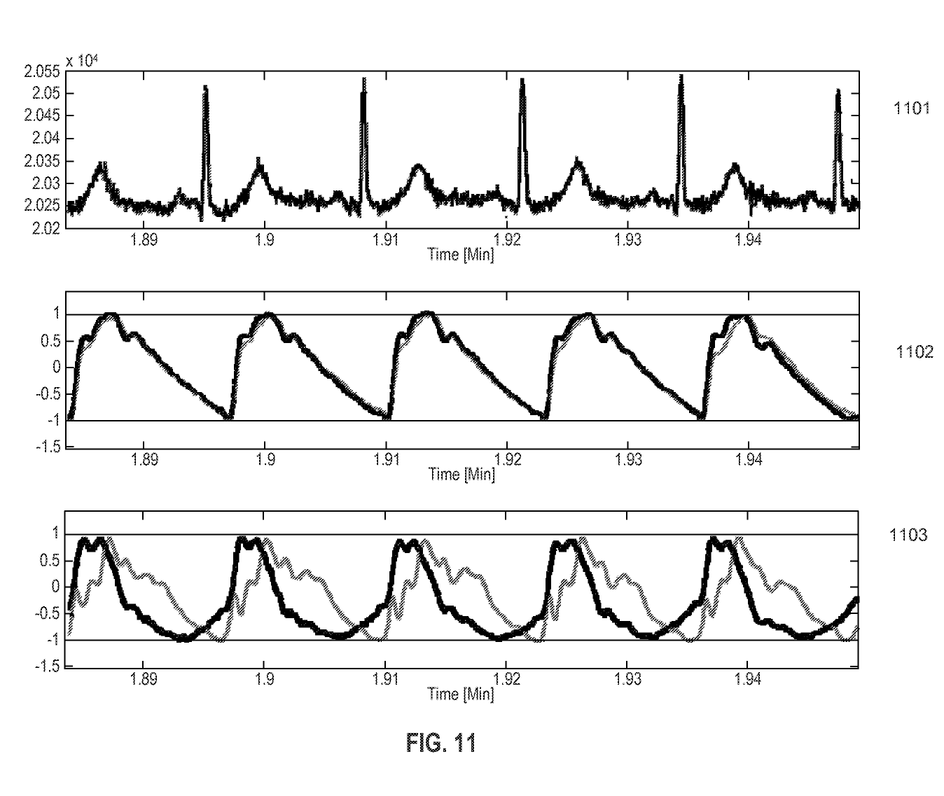

Impedance waveforms of patients suffering from vasospasm display differences from those of healthy patients. These differences may be used, through the apparatuses and method described herein, to detect, diagnose, and monitor vasospasm. FIG. 11 illustrates IPG waveforms recorded from a patient experiencing vasospasm. This patient experienced vasospasm 5 days after initial hospitalization from an aneurysm. Chart 1101 represents ECG, the chart 1102 represents impedance amplitude, and chart 1103 represents impedance phase. In the impedance charts 1102 and 1103, the dark lines represent the right hemisphere and the light lines represent the left. In chart 1103, it can be seen that certain parameters of the left hemisphere impedance phase (light line) are delayed with respect to the right (dark line). For example, the maximum slope in each cardiac cycle occurs later, as does the peak value in each cardiac cycle. Thus, a timing difference between a characteristic of a right hemisphere impedance waveform extracted from a right hemisphere signal and a corresponding characteristic of a left hemisphere impedance waveform extracted from a left hemisphere signal may be used to determine vasospasm. These parameters, and others, may be used to detect vasospasm.

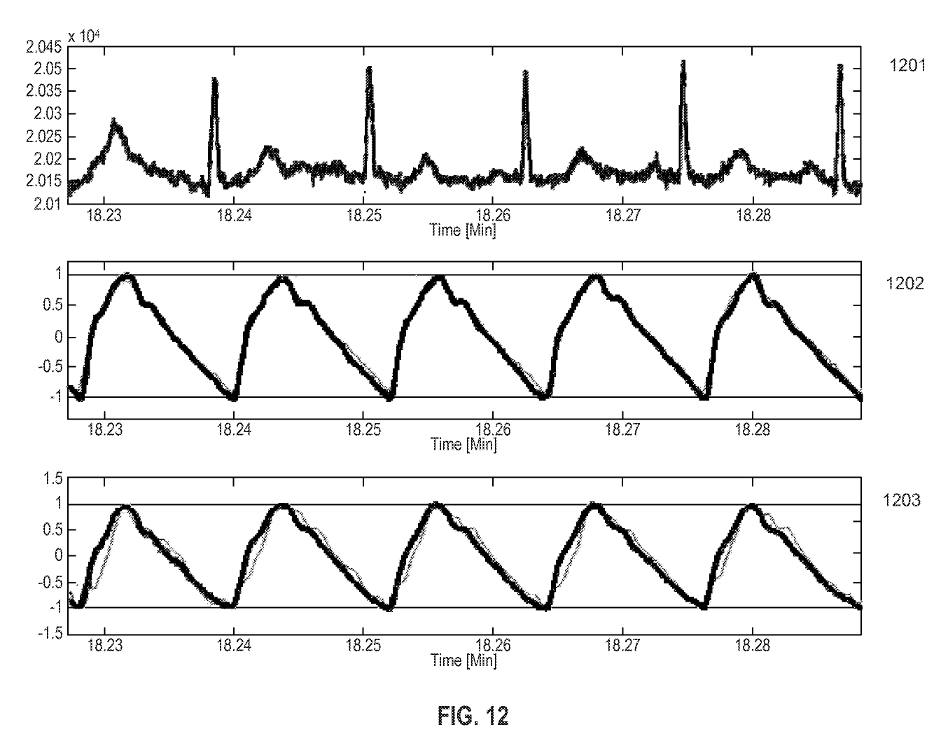

FIG. 12 shows recordings from the same patient, taken 3 minutes after the administration of nimotop. Chart 1201 represents ECG, the chart 1202 represents impedance amplitude, and chart 1203 represents impedance phase. In the impedance charts 1202 and 1203, the dark lines represent the right hemisphere and the light lines represent the left. As can be seen in the chart 1203, the timing between the impedance phase waveforms of the left and right hemispheres is more consistent after nimotop administration.

An IPG apparatus may be used to extract impedance waveforms from IPG signals and, based on parameters of the extracted waveforms, detect, diagnose, and monitor vasospasm in a subject.

Another embodiment consistent with the present disclosure includes an emergency traumatic brain injury monitor. In many situations, such as in battlefield hospitals, ambulances, emergency rooms, and sporting events, it may be important to obtain an early diagnosis of brain damage level during the initial diagnosis phase prior to transferring the patient to more specialized facilities in which imaging techniques such as CT and/or MRI may be applied.

Such early diagnoses may be of help in triage, i.e., determining which patients should be immediately transferred and others which may not have experienced brain damage. A diagnosis monitor, such as cerebral perfusion monitor 130, utilizing IPG may be used to determine the existence of damage to at least one of brain or blood brain barrier (BBB). If the presence of brain or BBB damage is detected, the IPG measurement apparatus may estimate a level of traumatic brain injury (TBI) (e.g., none, mild, or severe) and a level or extent of damage to a brain or BBB.

Such a diagnosis monitor may include at least one processor, as described herein, configured to perform signal processing and analysis on IPG data obtained from a subject. IPG data may be obtained through the use of one or more current delivery electrodes and one or more voltage sensing electrodes. Various electrode configurations may provide suitable IPG measurement results. In one embodiment, one pair of current delivery/voltage sensing electrodes is provided on one side of the head, and a second pair is provided on the other side of the head. The included processor may send signals to the electrodes and receive at least one IPG signal associated with the brain of the subject.

The processor may be configured to extract at least one cardiac pulsatility waveform from the IPG signal and at least one static value waveform from the IPG signal. The processor may extract at least one dynamic parameter from the cardiac pulsatility waveform, such as those previously discussed, e.g., a peak to peak measure, a rise time measure, or any other parameter associated with a cardiac pulsatility waveform of an IPG signal as discussed herein. The processor may extract at least on static parameter from the static value waveform, including any parameter discussed herein with respect to static waveforms, such as peak to peak measure and others. The processor may analyze and compare the extracted dynamic and static parameters of the obtained IPG signals, for example using various techniques described herein, to estimate levels of TBI or damage to at least one of a brain or BBB. Parameters of the IPG signals may be compared across the hemispheres of the brain, parameters of the IPG signals may be compared to predetermined values, and parameters of the IPG signals may be compared to additional parameters from the same IPG signal.

TABLE 1

| | | | Static IPG | Static IPG |
|---|---|---|---|---|
| | | | value - | value - |
| | | Injury | injury side | opposite side |
| Patient # | TBI level | Side | [Ohm] | [Ohm] |
| 7026 | Severe | Bilateral | 80 | 80 |
| 7029 | Severe | Right | 90 | 145 |
| 7030 | Severe | Bilateral | 75 | 85 |
| 7032 | Severe | Right | 67 | 100 |
| 7033 | Severe | Left | 50 | 90 |
| 7034 | Severe | Right | 80 | 113 |
| 7035 | Severe | Bilateral | 70 | 90 |
| 7036 | Severe | Bilateral | 87 | 95 |
| 7037 | Severe | Right | 85 | 135 |
| 7039 | Severe | Bilateral | 110 | 105 |
| 7040 | Severe | Bilateral | 40 | 60 |
| 7041 | Severe | Left | 75 | 79 |
| 7048 | Severe | Bilateral | 105 | 108 |
| 7049 | Severe | Bilateral | 52 | 97 |
| 7050 | Severe | Left | 67 | 112 |
| 7051 | Severe | Right | 69 | 58 |
| 7052 | Severe | Right | 110 | 118 |
| 9016 | Severe | Bilateral | 97 | 111 |
| 9018 | Severe | Bilateral | 132 | 113 |
| 9019 | Severe | Bilateral | 110 | 125 |
| 9020 | Severe | Bilateral | 78 | 99 |
| 9022 | Severe | Left | 47 | 118 |
| 1029 | Healthy | None | 133 | 123 |
| 1030 | Healthy | None | 152 | 156 |
| 1031 | Healthy | None | 147 | 150 |
| 1033 | Healthy | None | 133 | 130 |
| 1034 | Healthy | None | 163 | 169 |
| 1036 | Healthy | None | 138 | 145 |
| 1037 | Healthy | None | 161 | 153 |
| 1038 | Healthy | None | 167 | 162 |

Table 1 illustrates an exemplary parameter comparison for the diagnosis of TBI. Shown are static IPG values obtained from both sides of a subject's head. As shown in the table, patients suffering from TBI display lower values of static IPG impedance, and those with unilateral TBI typically show a disparity between measurements of the right side and the left side. Measurement of static IPG values may thus provide valuable information for quickly and non-invasively diagnosing TBI. Other measures discussed herein may provide similarly valuable information.

In some embodiments, a TBI monitor consistent with the present disclosure may include a carrier configured to fit on a head of the subject for electrode positioning, such as headset apparatus 120. A carrier of the TBI monitor may also include more or fewer electrode pairs than described with respect to exemplary headset apparatus 120. The TBI monitor of the present disclosure may be configured for portability, for example by reducing the size of a cerebral perfusion monitor 130 and permitting a cerebral perfusion monitor 130 to receive power via a rechargeable battery

It will be understood by a person of skill in the art that the methods presented herein for determining ICP through IPG waveform analysis are not limited to the examples presented. For example, many of the analysis methods are equally suitable for identifying features and characteristics within an ABP signal or ECG signal that may aid in the estimation of ICP, when used alone or in conjunction with data obtained from an IPG signal.

While this disclosure provides examples of the analysis of IPG signals, any signal that characterizes at least one cranial bioimpedance measurement may be assessed consistent with broad principles of this disclosure. While exemplary methods techniques in this disclosure are provided with respect to estimates of intracranial pressure, these methods and techniques may be used or adapted for estimation of any intracranial hemodynamic parameters. Further, the disclosure of uses of embodiments of the disclosed embodiments for detection, diagnosis, and monitoring of the discussed intracranial hemodynamic parameters is exemplary only. In its broadest sense, the disclosed embodiments may be used in connection with the detection, diagnosis, monitoring, and/or treatment of any physiological brain condition detectable using the principles described herein. Alternative embodiments will become apparent to those skilled in the art without departing from its spirit and scope. Accordingly, the scope of the disclosed embodiments is defined by the appended claims rather than the foregoing description.

What is claimed is:

1. A cerebro-hemodynamic measurement apparatus, comprising:
at least one processor configured to:
receive, via at least one sensor, at least one signal associated with a brain of a subject;
determine based on the at least one signal, a change in cerebral blood volume from a cardiac pulsation;
determine, based on the at least one signal, a change in intracranial pressure due to the cardiac pulsation;
determine a compliance indicator from a static portion of the at least one signal; and
estimate a mean intracranial pressure based on the change in cerebral blood volume, the change in intracranial pressure, and the compliance indicator.

2. The apparatus of claim 1, wherein the at least one processor is further configured to determine the change in cerebral blood volume from a hemispherical signal.

3. The apparatus of claim 1, wherein the at least one processor is further configured to determine the change in intracranial pressure from a trans-hemispherical signal.

4. The apparatus of claim 1, wherein the at least one processor is further configured to determine the change in cerebral blood volume from a real component of the at least one signal.

5. The apparatus of claim 1, wherein the at least one processor is further configured to determine the change in intracranial pressure from an imaginary component of the at least one signal.

6. The apparatus of claim 1, wherein the at least one processor is further configured to determine the change in intracranial pressure and the change in cerebral blood volume from a peak to peak measurement of the at least one signal.

7. The apparatus of claim 1, wherein the at least one processor is further configured to determine the compliance indicator from the static portion of the at least one signal and a condition of a patient.

8. The apparatus of claim 7, wherein the condition of the patient includes at least one of age, gender, head circumference, weight, existence of traumatic brain injury, existence of surgical intervention, existence of hemorrhage, existence of edema, pulse rate, and an injury side.

9. The apparatus of claim 1, wherein the at least one signal corresponds to an impedance plethysmography signal.

10. The apparatus of claim 1, wherein the at least one processor is further configured to determine the change in cerebral blood volume from an impedance plethysmography signal and at least one arterial blood pressure signal.

11. A cerebro-hemodynamic measurement apparatus, comprising:
at least one processor configured to:
send signals to a first pair of electrodes attached to a carrier configured to fit on a first portion of a head of a subject;
receive at least one impedance plethysmography signal from a second pair of electrodes attached to a carrier configured to fit on a second portion of a head of a subject;
extract at least one cross impedance plethysmography waveform corresponding to the first and second portions of the head of the subject from the impedance plethysmography signal; and
estimate a mean intracranial pressure based on changes in the cross impedance plethysmography waveform.

12. The apparatus of claim 11, wherein the at least one processor is further configured to:
send a second at least one signal to one portion of the head from the first portion or the second portion;
receive a second at least one IPG signal from the one portion of the head,
extract at least one IPG waveform from the second at least one IPG signal; and
estimate the mean ICP based on changes in the at least one cross IPG waveform and the at least one IPG waveform.

13. The apparatus of claim 11, wherein the at least one processor is further configured to:
receive an arterial blood pressure signal, and
estimate the mean intracranial pressure based on changes in at least one cross impedance plethysmography waveform and the arterial blood pressure signal.

14. The apparatus of claim 11, wherein the at least one processor is further configured to:
receive a noninvasive blood pressure signal, and
estimate the mean intracranial pressure based on changes in the at least one cross impedance plethysmography waveform and the noninvasive blood pressure signal.

15. The apparatus of claim 12, wherein the at least one processor is further configured to:
send the second at least one signal to the first pair of electrodes located on the first portion of the head; and
receive the second at least one signal from a third pair of electrodes located on the first portion of the head.

16. A cerebro-hemodynamic measurement apparatus, comprising:
at least one processor configured to:
send signals to at least one pair of electrodes attached to a carrier configured to fit on a head of a subject;
receive at least one impedance plethysmography signal associated with a brain of the subject; and
estimate a level of damage to at least one of a brain or blood brain barrier using the impedance plethysmography signal.

17. The apparatus of claim 16, wherein the at least one processor is further configured to:
extract at least one cardiac pulsatility waveform from the impedance plethysmography signal;
extract at least one static value waveform from the impedance plethysmography signal;
extract at least one dynamic parameter characterizing the cardiac pulsatility waveform;
extract at least one static parameter characterizing the static value waveform; and
estimate the level of damage to at least one of a brain or blood brain barrier based on a comparison between the at least on dynamic parameter and the at least one static parameter.

18. The apparatus of claim 16, wherein the at least one pair of electrodes includes a first pair of electrodes including a first current delivery electrode and a first voltage sensing electrode, and a second pair of electrodes including a second current delivery electrode and a second voltage sensing electrode.

19. The apparatus of claim 16, wherein the first pair of electrodes are arranged on the carrier so as to contact a right side of the head of the subject, and the second pair of electrodes are arranged on the carrier so as to contact the left side of the head of the subject.

20. A cerebro-hemodynamic measurement apparatus, comprising:
at least one processor configured to:
receive, via at least a pair of electrodes, at least one signal associated with a brain of a subject;
extract at least one impedance waveform from the at least one signal associated with the brain of the subject; and
determine an occurrence of vasospasm based on the at least one impedance waveform.

21. The apparatus of claim 20, wherein the at least one impedance waveform includes an impedance amplitude and an impedance phase.

22. The apparatus of claim 20, wherein the at least one signal includes a right hemisphere signal from a right hemisphere of the brain of the subject and a left hemisphere signal from a left hemisphere of the brain of the subject.

23. The apparatus of claim 19, wherein vasospasm is detected based on a timing difference between a characteristic extracted from a right hemisphere impedance waveform extracted from a right hemisphere signal and a left hemisphere impedance waveform extracted from a left hemisphere signal.
